(19) United States
(12) Patent Application Publication
(10) Pub. No.: US 2012/0196339 A1
(43) Pub. Date: Aug. 2, 2012

(54) PRODUCTION OF INDUSTRIALLY RELEVANT COMPOUNDS IN PROKARYOTIC ORGANISMS

(75) Inventors: Andrew T. Koppisch, Flagstaff, AZ (US); David T. Fox, Los Alamos, NM (US); Kinya Hotta, Singapore (SG); John D. Welsh, Pennington, NJ (US)

(73) Assignee: Los Alamos National Security LLC

(21) Appl. No.: 13/018,066

(22) Filed: Jan. 31, 2011

(51) Int. Cl. C12P 7/48; C12P 7/22; C12P 7/44; C12P 7/42

(52) U.S. Cl. 435/144; 435/146; 435/156; 435/142

(57) ABSTRACT

Disclosed herein are methods for producing compounds (such as 3,4-dihydroxybenzoate, catechol, cis,cis-muconate, or β-carboxy-cis,cis-muconic acid) utilizing biosynthetic pathways in prokaryotic organisms expressing one or more heterologous genes. In some embodiments, the method includes expressing a heterologous asbF gene (for example, a gene having dehydroshikimate dehydratase activity) in a prokaryotic cell under conditions sufficient to produce the one or more compounds and purifying the compound. In additional embodiments, the method further includes expressing one or more of a heterologous 3,4-DHB decarboxylase gene, a heterologous catechol 1,2-dioxygenase gene, and a heterologous 3,4-DHB dioxygenase gene in the prokaryotic cell and purifying the compound.

### ACKNOWLEDGMENT OF GOVERNMENT SUPPORT

This invention was made with government support under Contract No. DE-AC52-06NA25396 awarded by the U.S. Department of Energy. The government has certain rights in the invention.

### FIELD

This disclosure relates to biosynthesis of compounds in prokaryotic organisms, in particular compounds derived from dehydroshikimate.

### BACKGROUND

Catechol and catechol-derived products are globally consumed commodities of importance to a wide range of industrial applications, including textile and pharmaceutical synthesis, pesticide production, and the specialty chemical industry. Catechol, like the majority of all phenol derivatives, is currently produced on an industrial scale (global consumption >20,000 metric tons per year) via distilling of thermally cracked crude oil, or by oxidation of benzene. Not only are these processes environmentally harmful, but production costs are dictated by the price of crude oil. In addition, industrial production of these chemicals frequently requires high temperatures and pressures, transition metal catalysts, nitric acid, and generates a significant amount of pollution.

An alternative to these processes is biosynthesis of desired compounds or their precursors. It would be additionally beneficial if the compounds are produced in a photosynthetic organism. This allows for a renewable production of commodity chemicals using a method that not only minimizes energy consumption for production, but removes and utilizes environmental CO₂.

### SUMMARY

Disclosed herein are methods for producing compounds (for example, commodity chemicals) utilizing biosynthetic pathways in a prokaryotic organism expressing one or more heterologous genes. In some examples, the compounds are derived from a biosynthetic pathway utilizing dehydroshikimate as a precursor and/or are compounds in the β-ketoadipate pathway. In some embodiments, the compounds include one or more of 3,4-dihydroxybenzoate (3,4-DHB), catechol, cis,cis-muconate, and β-carboxy-cis,cis-muconic acid.

In some embodiments, the method includes expressing a heterologous asbF gene (for example, a gene having dehydroshikimate dehydratase activity) in a prokaryotic cell under conditions sufficient to produce the one or more compounds and purifying the compound. In one example, the compound produced is 3,4-DHB. In some examples, the prokaryotic cell is a heterotroph, a mixotroph, or a phototroph. In particular examples, the prokaryotic organism is a heterotroph, (such as a bacterial cell, for example, E. coli or Bacillus sp.) or a phototroph (such as a cyanobacterial cell, for example, Synechocystis sp.). In some examples, the asbF gene is a Bacillus sp. asbF gene (for example, SEQ ID NOs: 1-3).

In another embodiment, the method includes expressing a heterologous asbF gene and a heterologous 3,4-DHB decarboxylase gene in the prokaryotic cell and purifying the compound. In one example, the compound produced is catechol. In some examples, the 3,4-DHB decarboxylase gene is from Klebsiella pneumoniae, Enterobacter cloacae, Lactobacillus plantarum, or Clostridium butryricum (for example, one of SEQ ID NOs: 4-11).

In a further embodiment, the method includes expressing a heterologous asbF gene, a heterologous 3,4-DHB decarboxylase gene, and a heterologous catechol 1,2-dioxygenase gene in a prokaryotic cell and purifying the compound. In one example, the compound produced is cis,cis-muconate. In one example, the catechol 1,2-dixoygenase gene is from Streptomyces sp. 2065 (for example, SEQ ID NOs: 12-15). In some examples, the method further includes converting the cis,cis-muconic acid to adipic acid.

In another embodiment, the method includes expressing a heterologous asbF gene and a heterologous 3,4-DHB dioxygenase gene in a prokaryotic cell and purifying the compound. In one example, the compound is β-carboxy-cis,cis-muconic acid. In one example, the 3,4-DHB dioxygenase gene is from Streptomyces sp. 2065 (for example, SEQ ID NOs: 16-19. In some examples, the method further includes converting the β-carboxy-cis,cis-muconate to β-carboxy adipic acid.

The foregoing and other features of the disclosure will become more apparent from the following detailed description, which proceeds with reference to the accompanying figures.

### BRIEF DESCRIPTION OF THE DRAWINGS

FIG. 1A is a diagram of an exemplary biosynthetic pathway producing 3,4-DHB, catechol, cis,cis-muconic acid, and adipic acid.

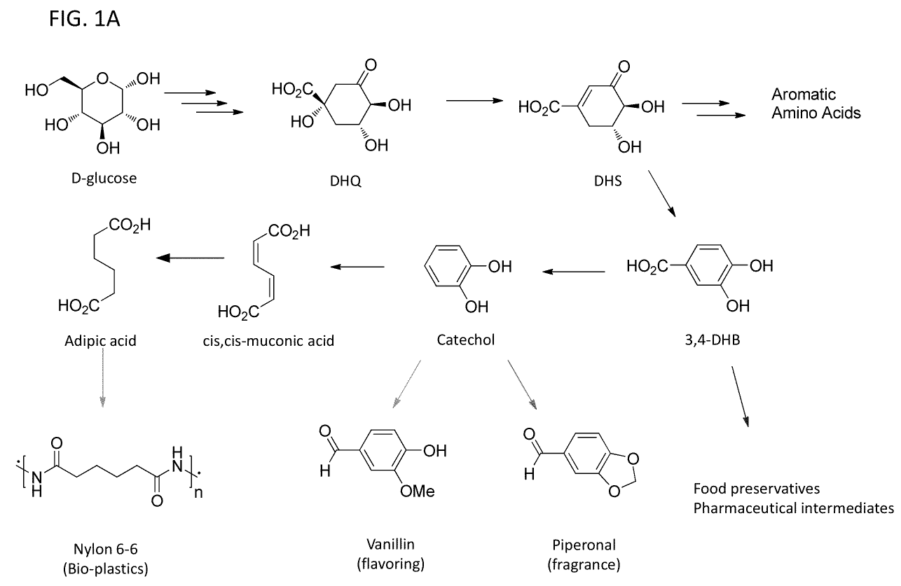

FIG. 1B is a diagram of an exemplary biosynthetic pathway producing β-carboxy-cis,cis-muconic acid from 3,4-DHB utilizing a 3,4-DHB dioxygenase.

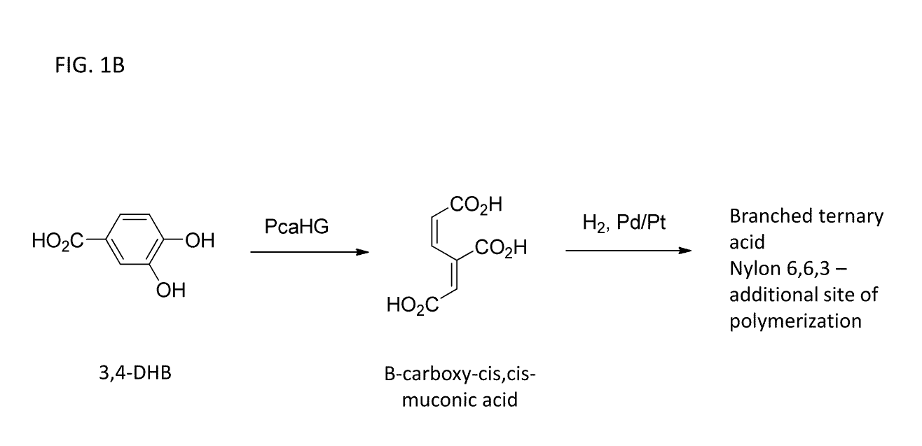

FIG. 2 is a phylogenetic tree of amino acid sequences including bacterial DHS dehydratases in GenBank and AsbF from B. thuringiensis 97-27 subsp. konkukian.

FIG. 3 is a digital image of gel electrophoresis of protein extract from E. coli expressing asbF and a Clostridium butyricum 3,4-DHB decarboxylase (left) or asbF and an Enterobacter cloacae 3,4-DHB decarboxylase (right). The upper boxed band (about 50 kDa) is the 3,4-DHB decarboxylase protein and the lower boxed band (about 35 kDa) is the AsbF protein.

FIG. 4 shows UV-Vis spectroscopy of a catechol standard (upper left panel) and catechol isolated from E. coli expressing asbF and 3,4-DHB decarboxylase (middle left panel), thin layer chromatography of catechol isolated from E. coli expressing asbF and a Clostridium butyricum 3,4-DHB decarboxylase (left) or asbF and an Enterobacter cloacae 3,4-DHB decarboxylase (right) (lower left panel), and ¹H NMR spectra of catechol isolated from induced or uninduced cells (right panels).

FIG. 5 is a diagram showing a flow cytometer isolating singular cells through hydrodynamic focusing, and the resulting projections of the complied data after 10,000 cells have been analyzed.

FIG. 6 is a graph showing OD₆₈₅ readings over a three week period of T1, T2, and T3 PCC 6803 cultures.

FIG. 7 is a graph showing auto-fluorescence readings for T1, T2, and T3 PCC 6803 cultures over a three week period.

FIG. 8 is a graph showing auto-fluorescence for each culture over the logarithmic growth period (days 6-10).

FIG. 9A-C is a series of plots of fluorescent intensity of the T3 culture. FIG. 9A shows the initial fluorescence of the T3 population with the P3 and P4 gates indicated. FIG. 9B shows the initial fluorescence of the sorted P4 population. FIG. 9C shows the initial fluorescence of the sorted P3 population.

### SEQUENCE LISTING

The nucleic acid and amino acid sequences listed herein are shown using standard letter abbreviations for nucleotide bases, and one letter code for amino acids. Only one strand of each nucleic acid sequence is shown, but the complementary strand is understood as included by any reference to the displayed strand.

The Sequence Listing is submitted as an ASCII text file in the form of the file named Sequence_Listing.txt, which was created on Jan. 31, 2011, and is 103,878 bytes, which is incorporated by reference herein.

SEQ ID NOs: 1 and 3 are nucleic acid sequences of exemplary B. thuringiensis 97-27 asbF genes suitable for expression in E. coli and Synechocystis, respectively.

SEQ ID NO: 2 is the amino acid sequence of an exemplary B. thuringiensis 97-27 AsbF protein encoded by SEQ ID NOs: 1 and 3.

SEQ ID NOs: 4 and 5 are the nucleic acid and amino acid sequences, respectively, of an exemplary 3,4-DHB decarboxylase from Klebsiella pneumoniae.

SEQ ID NOs: 6 and 7 are the nucleic acid and amino acid sequences, respectively, of an exemplary 3,4-DHB decarboxylase from Enterobacter cloacae.

SEQ ID NOs: 8 and 9 are the nucleic acid and amino acid sequences, respectively, of an exemplary 3,4-DHB decarboxylase from Lactobacillus plantarum.

SEQ ID NOs: 10 and 11 are the nucleic acid and amino acid sequences, respectively, of an exemplary 3,4-DHB decarboxylase from Clostridium butyricum.

SEQ ID NOs: 12 and 13 are the nucleic acid and amino acid sequences, respectively, of an exemplary Acinetobacter radioresistens catechol 1,2-dioxygenase A subunit.

SEQ ID NOs: 14 and 15 are the nucleic acid and amino acid sequences, respectively, of an exemplary Acinetobacter radioresistens catechol 1,2-dioxygenase B subunit.

SEQ ID NOs: 16 and 17 are the nucleic acid and amino acid sequences, respectively, of an exemplary Streptomyces sp. 2065 3,4-DHB dioxygenase α subunit.

SEQ ID NOs: 18 and 19 are the nucleic acid and amino acid sequences, respectively, of an exemplary Streptomyces sp. 2065 3,4-DHB dioxygenase β subunit.

SEQ ID NO: 20 is the nucleic acid sequence of an exemplary vector for gene expression in cyanobacteria, encoding AsbF, 3,4-DHB decarboxylase, and catechol 1,2-dioxygenase proteins.

### DETAILED DESCRIPTION

### I. Abbreviations

asbF/AsbF petrobactin biosynthesis gene or protein, respectively

3,4-DHB 3,4-dihydroxybenzoate

DHS 3-dehydroshikimate

DHSase dehydroshikimate dehydratase

IPTG isopropyl β-D-1-thiogalactopyranoside

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. All sequence database accession numbers (such as GenBank, EMBL, or UniProt) mentioned herein are incorporated by reference in their entirety as present in the respective database on Jan. 31, 2011. In case of conflict, the present specification, including explanations of terms, will control. In addition, the materials, methods, and examples are illustrative only and not intended to be limiting.

In order to facilitate review of the various embodiments of the invention, the following explanations of specific terms are provided:

Adipic acid: A dicarboxylic acid having the following structure (CAS Ref. No. 124-04-9):

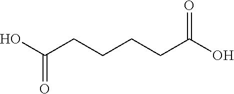

The major commercial use of adipic acid is as a monomer for the production of nylon and polyurethane. Adipic acid is also used as a flavorant or gelling aid in foods or pharmaceuticals.

AsbF: A petrobactin biosynthesis gene or protein (EC 4.2.1.118). The asbF gene encodes a protein having dehydroshikimate dehydratase (DHSase) activity, for example capable of catalyzing the transformation of 3-dehydroshikimate (DHS) to 3,4-dihydroxybenzoate (3,4-DHB). In some examples, the AsbF gene or protein is a Bacillus AsbF gene or protein, for example, from B. thuringiensis, B. cereus, or B. anthracis. In one non-limiting example, an asbF gene is from B. thuringiensis 97-27 (for example having the nucleic acid and amino acid sequences set forth in SEQ ID NOs: 1-3).

β-carboxy-cis,cis-muconic acid: A compound having the structure (CAS Reg.

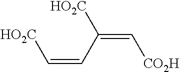

In some examples, β-carboxy-cis,cis-muconic acid can be synthesized directly from 3,4-DHB, for example by 3,4-DHB dioxygenase.

Catechol: Also known as pyrocatechol or 1,2-dihydroxybenzene (CAS Reg. No. 120-80-9). A compound having the structure:

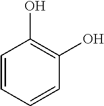

Catechol is utilized commercially in the production of pesticides and as a precursor to flavors (such as vanillin and ethylvanillin), fragrances (such as piperonal and 3-trans-isocamphylcyclohexanol), and pharmaceuticals.

Catechol 1,2-dioxygenase: An enzyme capable of catalyzing conversion of catechol to cis,cis-muconate (EC 1.13.11.1). Catechol 1,2-dioxygenase is a metalloproteinase that generally includes iron in the active site, although manganese-containing forms are known. These enzymes are primarily found in bacteria; however, fungal forms also exist. In particular examples, a catechol 1,2-dioxygenase gene is from a bacterium, such as Acinetobacter radioresistens or Herbaspirillum seropedicae (such as IsoA and/or IsoB). Catechol 1,2-dioxygenase as used herein refers to a nucleic acid or protein including two subunits (such as an A and a B subunit, two A subunits, or two B subunits).

Cis,cis-muconate: Also known as cis,cis-muconic acid (CAS Reg. No. 3588-17-8). A dicarboxylic acid having the structure:

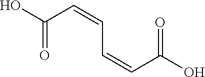

Cis,cis-muconate can be hydrogenated to adipic acid, for example by catalytic hydrogenation with platinum on carbon.

Conservative variants: A substitution of an amino acid residue for another amino acid residue having similar biochemical properties. “Conservative” amino acid substitutions are those substitutions that do not substantially affect or decrease an activity of a polypeptide (such as an AsbF polypeptide, a 3,4-DHB decarboxylase polypeptide, a 3,4-DHB dioxygenase polypeptide, or a catechol 1,2-dioxygenase polypeptide). A peptide can include one or more amino acid substitutions, for example 1-10 conservative substitutions, 2-5 conservative substitutions, 4-9 conservative substitutions, such as 1, 2, 5 or 10 conservative substitutions. Specific, non-limiting examples of a conservative substitution include the following examples (Table 1).

TABLE 1
Exemplary conservative amino acid substitutions

| Original Amino Acid | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The term conservative variation also includes the use of a substituted amino acid in place of an unsubstituted parent amino acid, provided that the substituted polypeptide retains an activity of the unsubstituted polypeptide. Thus, in one embodiment, non-conservative substitutions are those that reduce an activity of the polypeptide.

3-dehydroshikimate (DHS): Also known as 3-dehydroshikimic acid, 5-dehydroshikimate, or 5-dehydroshikimic acid (CAS Reg. No. 2922-42-1). A compound

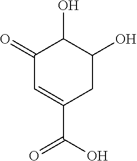

DHS is a precursor to aromatic amino acids, as well as catechol and cis,cis-muconic acid.

3,4-dihydroxybenzoate (3,4-DHB): Also known as protocatechuate or protocatechuic acid (CAS Reg. No. 99-50-3). A compound having the structure:

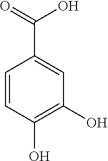

DHB is utilized commercially in the production of food preservatives and pharmaceutical intermediates.

Dihydroxybenzoate decarboxylase: Also known as protocatechuate decarboxylase (EC 4.1.1.63). 3,4-DHB decarboxylase catalyzes the conversion of 3,4-DHB to catechol. In some examples, a 3,4-DHB decarboxylase gene is from a bacterium, such as Enterobacter cloacae or Klebsiella pneumoniae.

3,4-Dihydroxybenzoate dioxygenase: Also known as protocatechuate dioxygenase (EC 1.13.11.3). 3,4-DHB dioxygenase catalyzes the direct conversion of 3,4-DHB to β-carboxy-cis,cis muconate. In some examples two subunits are required for 3,4-DHB dioxygenase activity, an α and a β subunit (e.g., pcaG and pcaH or pcaGH). In other examples, a homodimer of α subunits or a homodimer of α subunits can also have 3,4-DHB dioxygenase activity. 3,4-DHB dioxygenase as used herein refers to a nucleic acid or protein including two subunits (such as an α and a α subunit, two αsubunits, or two α subunits). In some examples, a 3,4-DHB dioxygenase gene is from a bacterium, such as Pseudomonas (for example, P. putida), Streptomyces, or Acinetobacter.

Expression: Transcription or translation of a nucleic acid sequence. For example, a gene is expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. The term “expression” is used herein to denote either transcription or translation. Regulation of expression can include controls on transcription, translation, RNA transport and processing, degradation of intermediary molecules such as mRNA, or through activation, inactivation, compartmentalization or degradation of specific protein molecules after they are produced.

Gene: A segment of nucleic acid that encodes an individual protein or RNA molecule (also referred to as a “coding sequence” or “coding region”) and may include non-coding regions (“introns”) and/or associated regulatory regions such as promoters, operators, terminators and the like, that may be located upstream or downstream of the coding sequence.

Heterologous: Originating from a different genetic sources or species. A gene that is heterologous to a prokaryotic cell originates from an organism or species other than the prokaryotic cell in which it is expressed. In one specific, non-limiting example, a heterologous asbF gene includes an asbF gene from Bacillus which is expressed in another bacterial cell (for example an E. coli cell) or which is expressed in a cyanobacterial cell (such as a Synechocystis cell). Methods for introducing a heterologous gene in a cell or organism are well known in the art, for example transformation with a nucleic acid, including electroporation, lipofection, and particle gun acceleration.

Heterotroph: An organism that cannot fix carbon and utilizes organic compounds as a carbon source. In some examples, a heterotroph is a prokaryotic heterotroph, such as a bacterium. In specific examples, a heterotrophic bacterium includes E. coli.

Isolated: An “isolated” biological component (such as a nucleic acid molecule, protein, or cell) has been substantially separated or purified away from other biological components in the cell of the organism, or the organism itself, in which the component naturally occurs, such as other chromosomal and extra-chromosomal DNA and RNA, proteins and cells. Nucleic acid molecules and proteins that have been “isolated” include nucleic acid molecules and proteins purified by standard purification methods. The term also embraces nucleic acid molecules and proteins prepared by recombinant expression in a host cell as well as chemically synthesized nucleic acid molecules and proteins. In other examples, the term includes small organic molecules, such as 3,4-DHB, catechol, cis,cis-muconate, and β-carboxy-cis,cis-muconic acid.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame. In some examples, a promoter sequence is operably linked to a protein encoding sequence, such that the promoter drives transcription of the linked nucleic acid and/or expression of the protein.

Phototroph: An organism that carries out photosynthesis to acquire energy. Phototrophs can utilize energy from light to convert carbon dioxide and water to compounds that can be used in cellular functions such as respiration and biosynthesis. In some examples, a phototroph is an obligate phototroph. In some examples, a phototroph is a prokaryotic phototroph, such as a cyanobacterium. In specific examples, a phototrophic cyanobacterium includes Synechocystis (such as Synechocystis PCC6803).

Prokaryotic cell: A cell or organism lacking a distinct nucleus or other membrane-bound organelles. Prokaryotes include the bacteria and archaea. In particular examples, prokaryotic cells include gram-positive bacteria, gram-negative bacteria (such as E. coli) and cyanobacteria (such as Synechocystis). Prokaryotic cells of use in the methods disclosed herein include those that can be transformed with and express heterologous genes.

Promoter: Promoters are sequences of DNA near the 5′ end of a gene that act as a binding site for RNA polymerase, and from which transcription is initiated. A promoter includes necessary nucleic acid sequences near the start site of transcription, such as, in the case of a polymerase II type promoter, a TATA element. In one embodiment, a promoter includes an enhancer. In another embodiment, a promoter includes a repressor element.

Promoters may be constitutively active, such as a promoter that is continuously active and is not subject to regulation by external signals or molecules. In some examples, a constitutive promoter is active such that expression of a sequence operably linked to the promoter is expressed ubiquitously (for example, in all cells of a tissue or in all cells of an organism and/or at all times in a single cell or organism, without regard to temporal or developmental stage).

Promoters may be inducible or repressible, such that expression of a sequence operably linked to the promoter can be expressed under selected conditions. In some examples, a promoter is an inducible promoter, such that expression of a sequence operably linked to the promoter is activated or increased. An inducible promoter may be activated by presence or absence of a particular molecule, for example, tetracycline, metal ions, alcohol, or steroid compounds. An inducible promoter also includes a promoter that is activated by environmental conditions, for example, light or temperature. In further examples, the promoter is a repressible promoter such that expression of a sequence operably linked to the promoter can be reduced to low or undetectable levels, or eliminated. A repressible promoter may be repressed by direct binding of a repressor molecule (such as binding of the trp repressor to the trp operator in the presence of tryptophan). In a particular example, a repressible promoter is a tetracycline repressible promoter. In other examples, a repressible promoter is a promoter that is repressible by environmental conditions, such as hypoxia or exposure to metal ions.

Purified: The term purified does not require absolute purity; rather, it is intended as a relative term. Thus, for example, a purified preparation of a compound is one in which the specified compound (such as 3,4-DHB, catechol, cis,cis-muconate, or β-carboxy-cis,cis-muconic acid) is more enriched than it is in its generative environment, for instance in a prokaryotic cell or in a cell culture (for example, in cell culture medium). Preferably, a preparation of a specified compound is purified such that the compound represents at least 50% of the total content of the preparation. In some embodiments, a purified preparation contains at least 60%, at least 70%, at least 80%, at least 85%, at least 90%, at least 95% or more of the specified compound.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith and Waterman (Adv. Appl. Math., 2:482, 1981); Needleman and Wunsch (J. Mol. Biol., 48:443, 1970); Pearson and Lipman (Proc. Natl. Acad. Sci., 85:2444, 1988); Higgins and Sharp (Gene, 73:237-44, 1988); Higgins and Sharp (CABIOS, 5:151-53, 1989); Corpet et al. (Nuc. Acids Res., 16:10881-90, 1988); Huang et al. (Comp. Appls. Biosci., 8:155-65, 1992); and Pearson et al. (Meth. Mol. Biol., 24:307-31, 1994). Altschul et al. (Nature Genet., 6:119-29, 1994) presents a detailed consideration of sequence alignment methods and homology calculations.

The alignment tools ALIGN (Myers and Miller, CABIOS 4:11-17, 1989) or LFASTA (Pearson and Lipman, Proc. Natl. Acad. Sci. 85:2444-2448, 1988) may be used to perform sequence comparisons. ALIGN compares entire sequences against one another, while LFASTA compares regions of local similarity. These alignment tools and their respective tutorials are available on the Internet. Alternatively, for comparisons of amino acid sequences of greater than about 30 amino acids, the “Blast 2 sequences” function can be employed using the default BLOSUM62 matrix set to default parameters, (gap existence cost of 11, and a per residue gap cost of 1). When aligning short peptides (fewer than around 30 amino acids), the alignment should be performed using the “Blast 2 sequences” function, employing the PAM30 matrix set to default parameters (open gap 9, extension gap 1 penalties). The BLAST sequence comparison system is available, for instance, from the NCBI web site; see also Altschul et al., J. Mol. Biol., 215:403-10, 1990; Gish and States, Nature Genet., 3:266-72, 1993; Madden et al., Meth. Enzymol., 266:131-41, 1996; Altschul et al., Nucleic Acids Res., 25:3389-402, 1997; and Zhang and Madden, Genome Res., 7:649-56, 1997.

Orthologs (equivalent to proteins of other species) of proteins are in some instances characterized by possession of greater than 75% sequence identity counted over the full-length alignment with the amino acid sequence of a specific protein using ALIGN set to default parameters. Proteins with even greater similarity to a reference sequence will show increasing percentage identities when assessed by this method, such as at least 80%, at least 85%, at least 90%, at least 92%, at least 95%, at least 98%, or at least 99% sequence identity.

When significantly less than the entire sequence is being compared for sequence identity, homologous sequences will typically possess at least 80% sequence identity over short windows of 10-20, and may possess sequence identities of at least 85%, at least 90%, at least 95%, 96%, 97%, 98%, or at least 99%, depending on their similarity to the reference sequence. Sequence identity over such short windows can be determined using LFASTA. One of skill in the art will appreciate that these sequence identity ranges are provided for guidance only; it is entirely possible that strongly significant homologs could be obtained that fall outside of the ranges provided. Similar homology concepts apply for nucleic acids as are described for protein. An alternative indication that two nucleic acid molecules are closely related is that the two molecules hybridize to each other under stringent conditions.

Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein.

Transduced and Transformed: A virus or vector “transduces” a cell when it transfers nucleic acid into the cell. A cell is “transformed” by a nucleic acid transduced into the cell when the DNA becomes stably replicated by the cell, either by incorporation of the nucleic acid into the cellular genome, or by episomal replication. As used herein, the term transformation encompasses all techniques by which a nucleic acid molecule is introduced into such a cell, including transformation with plasmid vectors, and introduction of naked DNA by electroporation, lipofection, and particle gun acceleration.

Vector: A nucleic acid molecule as introduced into a host cell (such as a prokaryotic cell), thereby producing a transformed host cell. A vector may include nucleic acid sequences that permit it to replicate in a host cell, such as an origin of replication. A vector may also include one or more selectable marker gene and other genetic elements known in the art. Vectors include plasmid vectors, including plasmids for expression in gram negative and gram positive bacterial cells. Exemplary vectors include those for expression in bacteria (such as E. coli) and cyanobacteria (such as Synechocystis).

### III. Overview of Several Embodiments

Disclosed herein are methods for producing compounds (such as industrially relevant compounds or commodity chemicals) in prokaryotic cells. The compounds synthesized utilizing the methods disclosed herein include compounds that are derived from dehydroshikimate as a precursor (either directly or indirectly). In some examples, the compounds are part of the β-ketoadipate biosynthetic pathway. In particular examples, the compounds include 3,4-DHB, catechol, cis,cis-muconate, adipic acid, β-carboxy-cis,cis-muconic acid, and β-carboxyadipic acid.

In some embodiments, the method includes expressing a heterologous asbF gene (for example, a gene having dehydroshikimate dehydratase activity) in a prokaryotic cell under conditions sufficient to produce the one or more compounds and purifying the compound. In one example, the compound produced is 3,4-DHB. In another embodiment, the method includes expressing a heterologous asbF gene and a heterologous 3,4-DHB decarboxylase gene in the prokaryotic cell and purifying the compound. In one example, the compound produced is catechol. In a further embodiment, the method includes expressing a heterologous asbF gene, a heterologous 3,4-DHB decarboxylase gene, and a heterologous catechol 1,2-dioxygenase gene in a prokaryotic cell and purifying the compound. In one example, the compound produced is cis,cis-muconate. In some examples, the method further includes converting the cis,cis-muconic acid to adipic acid. In another embodiment, the method includes expressing a heterologous asbF gene and a heterologous 3,4-DHB dioxygenase gene in a prokaryotic cell and purifying the compound. In some examples, the method further includes converting the β-carboxy-cis,cis-muconate to β-carboxy adipic acid.

In some embodiments, the prokaryotic cell does not include genetic modification of an endogenous gene. It has surprisingly been found that, utilizing the methods disclosed herein, in at least some examples, it is not necessary to modify the prokaryotic cell in order to redirect glucose metabolism to 3-dehydroshikimate, the precursor of 3,4-DHB. Therefore, in some examples, the prokaryotic cell does not include a mutation in an endogenous gene in the shikimate pathway (for example, a mutation in one or more endogenous genes which prevents conversion of 3-dehydroshikimate to chorismate).

Furthermore, use of a heterologous asbF gene in the methods disclosed herein decreases the problem of a 3,4-DHB “bottleneck” which limits the production of downstream compounds of interest, such as catechol, cis,cis-muconate, and β-carboxy-cis,cis-muconic acid. The inventors have identified AsbF as a particularly effective enzyme for producing 3,4-DHB in prokaryotic cells.

The disclosed methods include expressing one or more of the heterologous genes described herein in the prokaryotic cell under conditions sufficient to produce the desired compound. One of skill in the art can determine appropriate conditions to express the heterologous genes and produce the compounds, based on the particular genes, compounds, and cell utilized. In some examples, the conditions include culture conditions for the prokaryotic cell, including temperature, carbon source (for example, glucose) and concentration, and in the case of phototrophic cells, amount and wavelength of light exposure.

In some examples, conditions sufficient to produce the compound of interest are conditions wherein the cells expressing the one or more heterologous genes produces an increased yield of the compound (for example, at least 10% more, such as at least 20%, 30%, 40%, 50%, 60%, 70%, 80%, 90%, 100%, 200%, 300%, or more) compared to a control (such as cells not expressing the heterologous gene or cells cultured under non-optimized conditions). In other examples, conditions sufficient to produce the compound include conditions in which conversion of glucose to the compound of interest is at least about 50 μM/hour (such as about 100 μM/hour, 150 μM/hour, 200 μM/hour, 300 μM/hour, or more) or conditions in which crude yield of the compound from glucose is at least about 5% (such as about 10%, 15%, 20%, 25%, 30%, 40%, 50%, or more).

One of skill in the art can modify the culture conditions of the organism expressing an asbF gene to optimize production of the compound of interest. Conditions that can be modified for culture of heterotrophs or phototrophs include pH, temperature, glucose concentration (such as initial glucose concentration or addition of glucose during culture), and continuous extraction of the product (for example to minimize toxicity and/or to shift equilibrium to the product of interest). Additional conditions that can be modified for culture of phototrophs include carbon dioxide concentration, light-dark cycle times and light intensity. For either type of organism, the conditions are modified and product formation is measured to determine whether optimal conditions are achieved. However, it is to be understood that conditions sufficient to produce a product of interest do not require that production of the compound is optimal, merely that production occurs at a detectable level.

The disclosed methods include expression of one or more heterologous genes (discussed in detail below) in a prokaryotic cell. In some examples, the prokaryotic cell is a heterotroph, such as an organism that cannot fix carbon and utilizes organic compounds as a carbon source. In other examples, the prokaryotic cell is a phototroph, for example, an organism that utilizes energy from light to convert carbon dioxide and water to compounds that can be used in cellular functions such as respiration and biosynthesis. In further examples, the prokaryotic cell is a mixotroph, for example an organism that can utilize a mixture of sources of energy and carbon.

In some examples the prokaryotic organism is a heterotroph. In particular examples, the heterotroph is a bacterial cell. Suitable bacteria for the methods disclosed herein include but are not limited to Escherichia coli, Bacillus (such as B. brevis, B. cereus, B. circulans, B. coagulans, B. lichenformis, B. megaterium, B. mesentericum, B. pumilis, B. subtilis, or B. thuringiensis), Pseudomonas (such as P. putida, P. angulate, P. fluorescens, or P. tabaci), and Streptomyces (such as S. avermitilis, S. coelicolor, or S. lividans). In particular examples, the bacteria are E. coli, Bacillus (for example, members in the B. cereus sensu lato group), or Streptomyces (for example, S. coelicolor or S. lividans). One of skill in the art can identify additional bacteria suitable for use in the methods disclosed herein, such as bacteria amenable to genetic manipulation, for example expression of one or more heterologous genes.

In other examples the prokaryotic organism is a phototroph. In some examples, the phototroph is a cyanobacterial cell. Suitable cyanobacteria for the methods disclosed herein include Synechocystis sp. (e.g., Synechocystis PCC6803, Synechocystis PCC9714, Synechocystis 6714, Synechocystis PCC6308, Synechocystis PCC9413, or Synechocystis BO8402), Synechococcus sp. (for examples, Synechococcus PCC7942), Spirulina sp. (for example, Spirulina platensis), or Anabaena sp. (e.g., Anabaena variabilis). In a particular example, the cyanobacterial cell is Synechocystis PCC6803. One of skill in the art can identify additional cyanobacteria suitable for use in the methods disclosed herein, such as cyanobacteria amenable to genetic manipulation, for example, expression of one or more heterologous genes.

In further examples, the prokaryotic organism is a mixotroph. In one example, the mixotroph is able to utilize both glucose and CO₂ and light, such as Synechocystis PCC6803 with a disrupted PsbAII gene. In another example, the mixotroph is able to utilize either light and CO₂ under anaerobic conditions, or glucose under aerobic conditions in the dark such as the purple non-sulfur bacterium, Rhodobacter sphaeroides.

A. AsbF

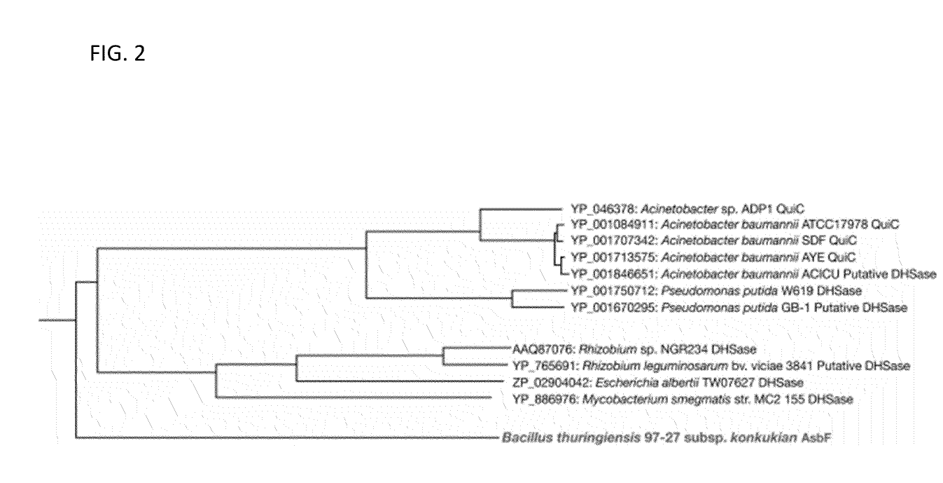

Specific disclosed methods include expressing a heterologous asbF gene in a prokaryotic cell under conditions sufficient to produce a compound of interest (such as a compound derived from dehydroshikimate, for example, 3,4-DHB, catechol, cis,cis-muconate, or β-carboxy-cis,cis-muconic acid). The asbF gene is a petrobactin biosynthesis gene and encodes a protein having dehydroshikimate dehydratase (DHSase) activity, for example capable of catalyzing the transformation of 3-dehydroshikimate (DHS) to 3,4-dihydroxybenzoate (3,4-DHB). The asbF gene is distinct from other know DHSases, having less than 50% sequence identity with previously identified DHSases (such as less than 45%, less than 40% less than 35%, less then 30% or less than 25% identity). Exemplary DHSases and their phylogenetic relationship with a B. thuringiensis AsbF are shown in FIG. 2.

In some examples, the asbF gene or protein is a Bacillus AsbF gene or protein, for example, from a member of the B. cereus sensu lato group (for example, B. thuringiensis, B. cereus, B. anthracis, or B. weihenstephanensis). In other examples, the AsbF gene or protein is an AsbF gene or protein from Streptomyces or Acinetobacter (such as Acinetobacter sp. strain ADP1, Acinetobacter sp. strain RUH2624, Acinetobacter sp. strain SH024, A. johnsonii, or A. baumanii). Nucleic acid and amino acid sequences for AsbF are publicly available. For example, GenBank Accession Nos. CP001903 (nucleotides 1893609-1894451), CP000485 (nucleotides 1916965-1917807), AE017355 (nucleotides 1908124-1908966), AE016877 (nucleotides 1932109-1932951), CP001176 (nucleotides 1902451-1903293), CP001186 (nucleotides 1863653-1864495), CP001746 (nucleotides 1841897-1842739), CP001283 (nucleotides 1927842-1928684), CP000001 (nucleotides 1927449-1928291), CP001407 (nucleotides 1906571-1907413), CP001598 (nucleotides 1870999-1871841), CP001215 (nucleotides 2368129-2367287), AE017334 (nucleotides 1871099-1871941), AE017225 (nucleotides 1871043-1871885), AE016879 (nucleotides 1870976-1871818), CP000903 (nucleotides 1920008-1920850), and EF038844 disclose exemplary asbF nucleic acid sequences. UniProt Accession Nos. Q813P6, B7HJA9, B71T99, C3P7H0, C3L5K5, Q81RQ4, B7JKH8, Q63CH2, C1ERB0, A0RCY9, Q6HJX7, and A9VRP6 and GenBank Accession No. Q43922 disclose exemplary AsbF amino acid sequences. Each of these sequences are incorporated by reference as provided by GenBank and/or UniProt databases on Jan. 31, 2011.

In one non-limiting example, an asbF gene is from B. thuringiensis 97-27. In some examples, the asbF gene includes or consists of the nucleic acid sequence set forth as:

(SEQ ID NO: 1)
ATGAAATATAGCCTGTGCACCATTAGCTTTCGTCATCAGCTGATTAGCTT
TACCGATATTGTGCAGTTCGCGTATGAAAACGGCTTTGAAGGCATTGAAC
TGTGGGGCACCCATGCGCAGAACCTGTATATGCAGGAATATGAAACCACC
GAACGTGAACTGAACTGCCTGAAAGATAAAACCCTGGAAATCACCATGAT
TAGCGATTATCTGGATATTAGCCTGAGCGCGGATTTTGAAAAAACCATCG
AAAAATGCGAACAGCTGGCCATTCTGGCCAACTGGTTCAAAACCAACAAA
ATTCGTACCTTTGCGGGCCAGAAAGGCAGCGCGGATTTCAGCCAGCAGGA
ACGTCAGGAATACGTTAACCGCATTCGCATGATTTGCGAACTGTTTGCGC
AGCATAACATGTATGTGCTGCTGGAAACCCATCCGAACACCCTGACCGAT
ACCCTGCCGAGCACCCTGGAACTGCTGGGCGAAGTGGATCATCCGAACCT
GAAAATCAACCTGGATTTTCTGCATATTTGGGAAAGCGGTGCCGATCCGG
TGGATAGCTTTCAGCAGCTGCGTCCGTGGATTCAGCATTACCACTTCAAA
AACATTAGCAGCGCCGATTATCTGCATGTGTTTGAACCGAACAACGTGTA
TGCGGCAGCGGGTAACCGTACCGGTATGGTGCCGCTGTTCGAAGGTATTG
TGAACTACGATGAAATCATTCAGGAAGTGCGCGATACCGATCATTTTGCG
AGCCTGGAATGGTTTGGCCATAACGCGAAAGATATTCTGAAAGCGGAAAT
GAAAGTGCTGACCAACCGTAACCTGGAAGTGGTGACCAGCTAG
(SEQ ID NO: 3)
AAATACTCCTTGTGCACCATTTCCTTTCGGCATCAATTGATTAGTTTTAC
CGATATTGTGCAATTTGCCTATGAAAATGGCTTTGAAGGCATTGAATTGT
GGGGCACCCATGCCCAAAATTTGTATATGCAAGAATATGAAACCACCGAA
CGGGAACTGAATTGCTTGAAAGATAAAACCTTGGAAATTACCATGATTTC
CGATTACCTGGACATTTCCTTGAGTGCCGATTTTGAAAAAACCATTGAAA
AATGTGAACAACTGGCCATTCTGGCCAATTGGTTTAAAACCAACAAAATT
CGGACCTTTGCCGGTCAAAAAGGCTCTGCCGATTTTTCCCAACAAGAACG
GCAAGAATACGTGAATCGGATTCGGATGATTTGTGAATTGTTTGCCCAGC
ATAACATGTATGTGTTGTTGGAAACCCATCCCAATACCTTGACCGATACC
TTGCCCTCCACCTTGGAATTGTTGGGCGAAGTGGATCATCCCAATCTGAA
AATTAACCTGGATTTTTTGCATATTTGGGAATCCGGTGCCGATCCCGTGG
ATTCCTTTCAACAATTGCGTCCCTGGATTCAACATTATCATTTTAAAAAT
ATTTCCAGTGCCGATTATTTGCATGTGTTTGAACCCAATAACGTGTATGC
CGCTGCCGGTAATCGGACCGGCATGGTGCCCTTGTTTGAAGGTATTGTGA
ACTATGATGAAATTATTCAAGAAGTGCGGGACACCGATCATTTTGCCAGT
TTGGAATGGTTTGGCCATAACGCCAAAGATATTTTGAAAGCCGAAATGAA
AGTGCTGACCAATCGGAATTTGGAAGTGGTGACCTCCTAA

In some embodiments, an asbF gene of use in the methods disclosed herein has a nucleic acid sequence at least 70%, 75%, 85%, 90%, 95%, 96%, 97%, 98% or 99% identical to the nucleic acid sequence set forth in SEQ ID NOs: 1 or 3. Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein.

In some examples, the asbF gene encodes a protein that includes or consists of the amino acid sequence set forth as:

(SEQ ID NO: 2)
MKYSLCTISFRHQLISFTDIVQFAYENGFEGIELWGTHAQNLYMQEYETT
ERELNCLKDKTLEITMISDYLDISLSADFEKTIEKCEQLAILANWFKTNK
IRTFAGQKGSADFSQQERQEYVNRIRMICELFAQHNMYVLLETHPNTLTD
TLPSTLELLGEVDHPNLKINLDFLHIWESGADPVDSFQQLRPWIQHYHFK
NISSADYLHVFEPNNVYAAAGNRTGMVPLFEGIVNYDEIIQEVRDTDHFA
SLEWFGHNAKDILKAEMKVLTNRNLEVVTS

In some embodiments, the polypeptide encoded by the asbF gene has an amino acid sequence at least 80%, 85%, 90%, 95%, 96%, 97%, 98% or 99% identical to the amino acid sequence set forth in SEQ ID NO: 2.

Exemplary nucleic acid and amino acid sequences can be obtained using computer programs that are readily available on the internet and the amino acid sequences set forth herein. In one example, the AsbF polypeptide retains a function of the AsbF protein, such as DHSase activity. Thus, a specific, non-limiting example of an AsbF polypeptide is a conservative variant of the AsbF polypeptide (such as a single conservative amino acid substitution, for example, one or more conservative amino acid substitutions, for example 1-10 conservative substitutions, 2-5 conservative substitutions, 4-9 conservative substitutions, such as 1, 2, 5 or 10 conservative substitutions). A table of conservative substitutions is provided above (Table 1).

B. 3,4-DHB Decarboxylase

Some embodiments of the disclosed methods include expressing a heterologous 3.4-DHB decarboxylase gene in a prokaryotic cell, for example, in addition to expressing a heterologous asbF gene. Expression of an asbF gene and a 3,4-DHB decarboxylase gene in a prokaryotic cell results in production of catechol by the cell when it is cultured under conditions sufficient to produce catechol. 3,4-DHB decarboxylase is also known as protocatechuate decarboxylase and has the Enzyme Commission (EC) number EC 4.1.1.63. 3,4-DHB decarboxylase catalyzes the conversion of 3,4-DHB to catechol.

In some examples, the 3,4-DHB decarboxylase gene or protein is a bacterial 3,4-DHB decarboxylase gene or protein, for example, from Enterobacter cloacae, Klebsiella pneumoniae, Lactobacillus plantarum, or Clostridium butyricon.

Nucleic acid and amino acid sequences for 3,4-DHB decarboxylase are publicly available. For example, GenBank Accession Nos. NZ_ACZD01000147 (nucleotides 16882-18390), AB364296, NZ_ACGZ02000022 (nucleotides 89805-91286), and NZ_ABDT01000049 (nucleotides 109154-110617) disclose exemplary 3,4-DHB decarboxylase nucleic acid sequences and GenBank Accession Nos. ZP_{—}06016267, BAG24502, ZP_{—}07078673, and ZP_{—}02948872 disclose exemplary 3,4-DHB decarboxylase amino acid sequences. Each of these sequences is incorporated by reference as provided by GenBank on Jan. 31, 2011. In a particular example, the 3,4-DHB decarboxylase gene is from Klebsiella pneumoniae, for example, the AroY gene (such as GenBank Accession No. AB479384). In another particular example, the 3,4-DHB decarboxylase gene is from Lactobacillus plantarum (such as L. plantarum subsp. plantarum ATCC 14917), for example, GenBank Accession No. AB364296. In some examples, the 3,4-DHB decarboxylase gene includes or consists of the nucleic acid sequence set forth as:

(SEQ ID NO: 4)
ATGACCGCACCGATTCAGGATCTGCGCGACGCCATCGCGCTGCTGCAACA
GCATGACAATCAGTATCTCGAAACCGATCATCCGGTTGACCCTAACGCCG
AGCTGGCCGGTGTTTATCGCCATATCGGCGCGGGCGGCACCGTGAAGCGC
CCCACCCGCATCGGGCCGGCGATGATGTTTAACAATATTAAGGGTTATCC
ACACTCGCGCATTCTGGTGGGTATGCACGCCAGCCGCCAGCGGGCCGCGC
TGCTGCTGGGCTGCGAAGCCTCGCAGCTGGCCCTTGAAGTGGGTAAGGCG
GTGAAAAAACCGGTCGCGCCGGTGGTCGTCCCGGCCAGCAGCGCCCCCTG
CCAGGAACAGATCTTTCTGGCCGACGATCCGGATTTTGATTTGCGCACCC
TGCTTCCGGCGCACACCAACACCCCTATCGACGCCGGCCCCTTCTTCTGC
CTGGGCCTGGCGCTGGCCAGCGATCCCGTCGACGCCTCGCTGACCGACGT
CACCATCCACCGCTTGTGCGTCCAGGGCCGGGATGAGCTGTCGATGTTTC
TTGCCGCCGGCCGCCATATCGAAGTGTTTCGCCAAAAGGCCGAGGCCGCC
GGCAAACCGCTGCCGATAACCATCAATATGGGTCTCGATCCGGCCATCTA
TATTGGCGCCTGCTTCGAAGCCCCTACCACGCCGTTCGGCTATAATGAGC
TGGGCGTCGCCGGCGCGCTGCGTCAACGTCCGGTGGAGCTGGTTCAGGGC
GTCAGCGTCCCGGAGAAAGCCATCGCCCGCGCCGAGATCGTTATCGAAGG
TGAGCTGTTGCCTGGCGTGCGCGTCAGAGAGGATCAGCACACCAATAGCG
GCCACGCGATGCCGGAATTTCCTGGCTACTGCGGCGGCGCTAATCCGTCG
CTGCCGGTAATCAAAGTCAAAGCAGTGACCATGCGAAACAATGCGATTCT
GCAGACCCTGGTGGGACCGGGGGAAGAGCATACCACCCTCGCCGGCCTGC
CAACGGAAGCCAGTATCTGGAATGCCGTCGAGGCCGCCATTCCGGGCTTT
TTACAAAATGTCTACGCCCACACCGCGGGTGGCGGTAAGTTCCTCGGGAT
CCTGCAGGTGAAAAAACGTCAACCCGCCGATGAAGGCCGGCAGGGGCAGG
CCGCGCTGCTGGCGCTGGCGACCTATTCCGAGCTAAAAAATATTATTCTG
GTTGATGAAGATGTCGACATCTTTGACAGCGACGATATCCTGTGGGCGAT
GACCACCCGCATGCAGGGGGACGTCAGCATTACGACAATCCCCGGCATTC
GCGGTCACCAGCTGGATCCGTCCCAGACGCCGGAATACAGCCCGTCGATC
CGTGGAAATGGCATCAGCTGCAAGACCATTTTTGACTGCACGGTCCCCTG
GGCGCTGAAATCGCACTTTGAGCGCGCGCCGTTTGCCGACGTCGATCCGC
GTCCGTTTGCACCGGAGTATTTCGCCCGGCTGGAAAAAAACCAGGGTAGC
GCAAAATAA
(SEQ ID NO: 6)
ACGCATCAGACGAAATTGCATGACGAAGTCCCGCGAATTTGATAATAAAA
TTCTATCAAAATAGCATCAATGATGCAATTGATGCTATCTGTCGTTCGCC
CAACAATGGAGGTCAGCCATTAAGGGAGAAAAACATGCAAAACCCCATCA
ACGATCTCAGAAGCGCCATCGCGTTGCTGCAACGCCATCCAGGTCACTAT
ATCGAAACCGATCACCCGGTAGATCCCAATGCTGAACTGGCGGGCGTCTA
CCGCCATATCGGCGCGGGCGGTACCGTAAAACGCCCCACCCGCACGGGCC
CGGCCATGATGTTCAATAGCGTGAAGGGCTACCCTGGCTCCCGCATCCTG
GTAGGTATGCACGCCAGCCGGGAAAGAGCGGCGCTTCTGCTGGGCTGTGT
ACCCTCGAAGCTGGCACAGCACGTTGGTCAGGCGGTGAAAAACCCGGTTG
CACCGGTGGTGGTTCCGGCCTCGCAGGCACCGTGCCAGGAGCAGGTCTTT
TACGCCGACGATCCGGACTTTGACCTGCGTAAGCTGCTTCCGGCCCCGAC
CAACACGCCGATTGATGCAGGCCCGTTCTTCTGTCTGGGGCTGGTACTGG
CAAGCGATCCGGAAGATACCTCGCTGACCGATGTGACCATTCACCGTCTC
TGTGTGCAGGAGCGAGATGAACTCTCTATGTTCCTTGCCGCCGGCCGCCA
TATCGAAGTCTTTCGCAAGAAGGCCGAAGCGGCGGGCAAACCGCTGCCGG
TAACCATCAATATGGGACTTGACCCGGCTATCTACATTGGGGCCTGTTTC
GAAGCGCCAACCACGCCATTCGGTTACAACGAGCTTGGCGTTGCCGGGGC
ATTACGCCAGCAACCGGTGGAGCTGGTACAGGGCGTGGCGGTAAAAGAGA
AAGCGATCGCGCGGGCGGAAATCATCATCGAGGGCGAACTGCTTCCCGGC
GTGCGCGTAAGAGAAGATCAGCACACCAACACCGGCCACGCCATGCCGGA
GTTCCCGGGCTACTGCGGCGAGGCGAATCCGTCTCTGCCGGTGATCAAAG
TGAAAGCCGTGACGATGCGAAACCACGCGATCCTGCAGACGCTGGTGGGC
CCGGGCGAAGAGCACACCACGCTTGCCGGTTTGCCGACCGAGGCCAGCAT
TCGCAACGCGGTCGAAGAGGCCATTCCCGGCTTTCTGCAAAACGTTTACG
CCCACACCGCCGGAGGCGGTAAATTCCTCGGCATTTTACAGGTGAAAAAA
CGCCAGCCGTCAGACGAAGGACGTCAGGGCCAGGCGGCACTTATCGCCCT
GGCCACCTATTCCGAGCTGAAAAACATTATCCTCGTGGATGAAGACGTGG
ATATCTTCGACAGCGACGATATCCTGTGGGCAATGACCACCCGCATGCAG
GGCGATGTGAGCATCACCACGCTTCCGGGGATCCGCGGCCACCAGCTGGA
TCCGTCGCAGTCACCGGACTACAGCACCTCGATCCGTGGAAACGGCATTT
CCTGCAAGACTATCTTCGACTGCACGGTGCCGTGGGCGCTGAAGGCGCGG
TTTGAACGGGCGCCGTTCATGGAGGTTGACCCCACACCGTGGGCGCCGGA
GCTGTTCAGCGATAAAAAATAGACCGTCGTCGCCGTTTCTTCGCCCCACC
GGGTGAAGAAACGCAAG
(SEQ ID NO: 8)
ATGAATGAAATGGCAGAACAACCATGGGATTTGCGTCGCGTGCTTGATGA
GATCAAGGATGATCCAAAGAACTATCATGAAACTGACGTCGAAGTTGATC
CAAATGCGGAACTTTCTGGTGTTTATCGGTATATCGGTGCTGGTGGGACC
GTTCAACGGCCAACGCAAGAGGGTCCAGCAATGATGTTTAACAACGTTAA
GGGGTTTCCTGATACGCGGGTCTTGACTGGATTGATGGCGAGTCGCCGGC
GCGTTGGTAAGATGTTCCACCACGATTATCAGACGTTAGGGCAATACTTG
AACGAAGCAGTCTCTAATCCAGTGGCGCCAGAAACGGTTGCTGAAGCGGA
TGCGCCAGCTCACGATGTCGTTTATAAAGCGACGGATGAAGGCTTTGATA
TTCGTAAGTTAGTGGCAGCACCAACGAATACGCCCCAAGATGCTGGACCA
TATATTACGGTCGGTGTGGTGTTTGGCTCAAGCATGGACAAGTCTAAGAG
TGATGTGACGATTCACCGAATGGTCCTTGAAGATAAGGATAAGTTAGGGA
TTTATATCATGCCTGGCGGTCGGCACATTGGTGCGTTTGCGGAAGAGTAT
GAGAAAGCTAACAAGCCAATGCCAATTACAATTAATATTGGTTTGGATCC
AGCCATTACGATTGGTGCAACTTTCGAACCACCGACCACGCCATTCGGTT
ATAACGAATTAGGTGTTGCTGGTGCGATTCGGAACCAAGCTGTTCAATTA
GTTGACGGGGTGACCGTCGATGAAAAGGCGATTGCGCGTTCTGAATATAC
GCTTGAGGGGTACATTATGCCTAACGAACGTATTCAGGAAGATATCAATA
CGCATACGGGCAAGGCGATGCCTGAATTCCCGGGTTATGATGGTGACGCC
AACCCAGCTTTACAAGTGATTAAGGTGACGGCGGTGACTCATCGGAAGAA
TGCCATCATGCAAAGCGTGATTGGACCATCCGAAGAACATGTCAGCATGG
CGGGAATTCCAACTGAAGCTAGTATCTTACAATTGGTTAACCGTGCCATT
CCTGGTAAAGTGACGAATGTTTATAATCCGCCGGCTGGTGGTGGTAAGTT
GATGACCATCATGCAGATTCACAAGGATAATGAAGCGGATGAAGGAATTC
AACGGCAAGCTGCCTTGCTTGCGTTCTCAGCCTTTAAGGAATTGAAGACT
GTTATCCTGGTTGATGAAGATGTTGATATTTTTGATATGAATGATGTGAT
TTGGACGATGAATACCCGTTTCCAAGCCGATCAGGACTTGATGGTCTTAT
CAGGCATGCGGAATCATCCGTTGGACCCATCGGAACGCCCACAATATGAT
CCAAAGTCGATTCGTTTCCGTGGGATGAGTTCTAAACTAGTGATTGATGG
CACCGTACCATTCGATATGAAGGACCAATTTGAACGGGCCCAATTCATGA
AAGTGGCTGACTGGGAGAAGTATTTGAAGTAA
(SEQ ID NO: 10)
ATGAGCAATAAAGTATATGATCTTAGAAGTGCATTAGAATTATTAAAAAC
TCTGCCAGGACAATTGATAGAAACAGATGTGGAAGTAGATTCAATGGCGG
AATTAGCAGGAGTTTATCGTTATGTTGGTGCTGGTGGAACGGTTCAGCGT
CCTACAAAAGAAGGACCAGCAATGATTTTTAATAATATAAAAGGACACAA
AGATGCAAGAGTATTAATTGGATTACTTGCAAGCCGTAGACGAGTGGCAG
CACTTTTAGATTGTGAACCTGAAAATTTAGGAAAGTTATTATATAGAAGT
GTCGATAATCCAATTGCCCCAGTACTTACAAACGCAAAATTACCTTTATG
TCAGCAGGTCGTTCATAAAGCAACAGATCCAGATTTTGATTTAAATAAAT
TAGTACCGGCACCAACAAATACACCTGATGATGCTGGGCCTTATATTACA
CTTGGAATGTGTTATGCAAGTCATCCAGATACAAAATTTAGTGATGTTAC
GATTCATCGTTTATGCATTCAGGGGAAGGATGAACTTTCAATATTCTTTA
CTCCAGGAGCAAGGCACATAGGTGCTATGGCAGAAAGAGCAGAAGAATTA
GGACAAAATCTTCCTATTTCAATAAGTATAGGTGTAGATCCTGCTATAGA
AATAGGTTCATGTTTTGAACCACCAACTACTCCATTAGGATATGATGAGT
TATCAGTTGCAGGAGCACTAAGAGGAAAGCCAGTGGAGCTTTGCAATTGT
ATTACAGTAAATGAAAGAGCTATTGCAAATGCCGAATATGTTATTGAAGG
TGAAGTTATACCTAATTTAAGAGTACAGGAAGATAAAAACAGCAATACAG
GATATGCTATGCCGGAATTTCCTGGGTATACAGGACCAGCAAGCGATCAA
TGTTGGATGATAAAGGTTAAAGCTGTTACACATAGAGAAAATCCAATTAT
GCAAACATGTATAGGTCCAAGTGAAGAGCACGTATCAATGGCAGGTATAC
CAACAGAAGCTAGTATTTATGGAATGATTGAAAAAGCAATGCCAGGAAGA
TTACAAAATGTATACTGCTGTTCATCTGGTGGTGGAAAATTCATGGCTGT
ATTACAGTTTAAAAAGACTGTTGCAAGTGATGAAGGGCGTCAAAGACAGG
CTGCATTATTAGCATTTTCAGCATTCAGTGAACTTAAAAATATATTCATT
GTAGATGAAGATGTGGACTGTTTTGATATGAATGATGTTTTATGGGCAAT
GAATACACGATTTCAGGGAGATGCAGATATTATAACAATTCCTGGAGTGA
GATGTCATCCACTTGATCCATCAAATGATCCAGATTATTCTCCAACCATA
AAAAATCATGGAATTGCATGTAAAACAATATTTGATTGTACTGTACCTTT
TCATATGAAAGAAAGATTTAAAAGAGCTAAATTTATGGAAGTTGATCCAG
AGCATTGGTTATAA

In some embodiments, a 3,4-DHB decarboxylase gene of use in the methods disclosed herein has a nucleic acid sequence at least 70%, 75%, 85%, 90%, 95%, 96%, 97%, 98% or 99% homologous to a nucleic acid sequence set forth in any one of SEQ ID NOs: 4, 6, 8, and 10. Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein.

In some embodiments, the 3,4-DHB decarboxylase gene encodes a protein that includes or consists of the amino acid sequence set forth as:

(SEQ ID NO: 5)
MTAPIQDLRDAIALLQQHDNQYLETDHPVDPNAELAGVYRHIGAGGTVKR
PTRIGPAMMFNNIKGYPHSRILVGMHASRQRAALLLGCEASQLALEVGKA
VKKPVAPVVVPASSAPCQEQIFLADDPDFDLRTLLPAHTNTPIDAGPFFC
LGLALASDPVDASLTDVTIHRLCVQGRDELSMFLAAGRHIEVFRQKAEAA
GKPLPITINMGLDPAIYIGACFEAPTTPFGYNELGVAGALRQRPVELVQG
VSVPEKAIARAEIVIEGELLPGVRVREDQHTNSGHAMPEFPGYCGGANPS
LPVIKVKAVTMRNNAILQTLVGPGEEHTTLAGLPTEASIWNAVEAAIPGF
LQNVYAHTAGGGKFLGILQVKKRQPADEGRQGQAALLALATYSELKNIIL
VDEDVDIFDSDDILWAMTTRMQGDVSITTIPGIRGHQLDPSQTPEYSPSI
RGNGISCKTIFDCTVPWALKSHFERAPFADVDPRPFAPEYFARLEKNQGS
AK
(SEQ ID NO: 7)
MQNPINDLRSAIALLQRHPGHYIETDHPVDPNAELAGVYRHIGAGGTVKR
PTRTGPAMMFNSVKGYPGSRILVGMHASRERAALLLGCVPSKLAQHVGQA
VKNPVAPVVVPASQAPCQEQVFYADDPDFDLRKLLPAPTNTPIDAGPFFC
LGLVLASDPEDTSLTDVTIHRLCVQERDELSMFLAAGRHIEVFRKKAEAA
GKPLPVTINMGLDPAIYIGACFEAPTTPFGYNELGVAGALRQQPVELVQG
VAVKEKAIARAEIIIEGELLPGVRVREDQHTNTGHAMPEFPGYCGEANPS
LPVIKVKAVTMRNHAILQTLVGPGEEHTTLAGLPTEASIRNAVEEAIPGF
LQNVYAHTAGGGKFLGILQVKKRQPSDEGRQGQAALIALATYSELKNIIL
VDEDVDIFDSDDILWAMTTRMQGDVSITTLPGIRGHQLDPSQSPDYSTSI
RGNGISCKTIFDCTVPWALKARFERAPFMEVDPTPWAPELFSDKK
(SEQ ID NO: 9)
MNEMAEQPWDLRRVLDEIKDDPKNYHETDVEVDPNAELSGVYRYIGAGGT
VQRPTQEGPAMMFNNVKGFPDTRVLTGLMASRRRVGKMFHHDYQTLGQYL
NEAVSNPVAPETVAEADAPAHDVVYKATDEGFDIRKLVAAPTNTPQDAGP
YITVGVVFGSSMDKSKSDVTIHRMVLEDKDKLGIYIMPGGRHIGAFAEEY
EKANKPMPITINIGLDPAITIGATFEPPTTPFGYNELGVAGAIRNQAVQL
VDGVTVDEKAIARSEYTLEGYIMPNERIQEDINTHTGKAMPEFPGYDGDA
NPALQVIKVTAVTHRKNAIMQSVIGPSEEHVSMAGIPTEASILQLVNRAI
PGKVTNVYNPPAGGGKLMTIMQIHKDNEADEGIQRQAALLAFSAFKELKT
VILVDEDVDIFDMNDVIWTMNTRFQADQDLMVLSGMRNHPLDPSERPQYD
PKSIRFRGMSSKLVIDGTVPFDMKDQFERAQFMKVADWEKYLK
(SEQ ID NO: 11)
MSNKVYDLRSALELLKTLPGQLIETDVEVDSMAELAGVYRYVGAGGTVQR
PTKEGPAMIFNNIKGHKDARVLIGLLASRRRVAALLDCEPENLGKLLYRS
VDNPIAPVLTNAKLPLCQQVVHKATDPDFDLNKLVPAPTNTPDDAGPYIT
LGMCYASHPDTKFSDVTIHRLCIQGKDELSIFFTPGARHIGAMAERAEEL
GQNLPISISIGVDPAIEIGSCFEPPTTPLGYDELSVAGALRGKPVELCNC
ITVNERAIANAEYVIEGEVIPNLRVQEDKNSNTGYAMPEFPGYTGPASDQ
CWMIKVKAVTHRENPIMQTCIGPSEEHVSMAGIPTEASIYGMIEKAMPGR
LQNVYCCSSGGGKFMAVLQFKKTVASDEGRQRQAALLAFSAFSELKNIFI
VDEDVDCFDMNDVLWAMNTRFQGDADIITIPGVRCHPLDPSNDPDYSPTI
KNHGIACKTIFDCTVPFHMKERFKRAKFMEVDPEHWL

Similarly, the polypeptide encoded by the 3,4-DHB decarboxylase gene can have an amino acid sequence at least 80%, 85%, 90%, 95%, 96%, 97%, 98% or 99% homologous to an amino acid sequences set forth as any one of SEQ ID NOs: 5, 7, 9, and 11.

In one example, the 3,4-DHB decarboxylase polypeptide retains a function of the wild-type protein, such as catalyzing conversion of 3,4-DHB to catechol. Thus, a specific, non-limiting example of a 3,4-DHB decarboxylase polypeptide is a conservative variant of the 3,4-DHB decarboxylase polypeptide (such as a single conservative amino acid substitution, for example, one or more conservative amino acid substitutions, for example 1-10 conservative substitutions, 2-5 conservative substitutions, 4-9 conservative substitutions, such as 1, 2, 5 or 10 conservative substitutions). A table of conservative substitutions is provided above (Table 1).

C. Catechol 1,2-dioxygenase

Additional embodiments of the disclosed methods include expressing a heterologous catechol 1,2-dioxygenase gene in a prokaryotic cell, for example, in addition to expressing a heterologous 3,4-DHB decarboxylase gene and a heterologous asbF gene. Expression of a catechol 1,2-dioxygense gene, a asbF gene, and a 3,4-DHB decarboxylase gene in a prokaryotic cell results in production of cis,cis-muconate by the cell when it is cultured under conditions sufficient to produce cis,cis-muconate. Catechol 1,2-dioxygenase (EC 1.13.11.1) catalyzes conversion of catechol to cis,cis-muconate.

In some examples, the catechol 1,2-dioxygenase gene or protein is a bacterial catechol 1,2-dioxygenase gene or protein, for example, from Acinetobacter (such as A. radioresistens, A. calcoaceticus or Acinetobacter sp. ADP1), Pseudomonas (such as P. putida), Burkholderia multivorans, or Herbaspirillum seropedicae. Nucleic acid and amino acid sequences for catechol 1,2-dioxygenase are publicly available. For example, GenBank Accession Nos. AF380158, AF182166, (nucleotides 807-1807), NZ_ACFD01000007 (nucleotides 29533-30468), NC_{—}014323 (nucleotides 1489376-1490311), NC_{—}005966 (nucleotides 1439848-1440783), NC_{—}002947 (nucleotides 4235833-4236768), and AY208917 (nucleotides 15119-16057, complement) disclose exemplary catechol 1,2-dioxygenase nucleic acid sequences and GenBank Accession Nos. AAK55425, AAG16896, ZP_{—}03573658, YP_{—}003777052, YP_{—}003774731, YP_{—}046127, and NP_{—}745846, disclose exemplary catechol 1,2-dioxygenase amino acid sequences. Each of these sequences is incorporated by reference as provided by GenBank on Jan. 31, 2011.

In a particular example, the catechol 1,2-dioxygenase gene is from Acinetobacter radioresistens, for example, the IsoA and/or IsoB genes. In some examples, the catechol 1,2-dioxygenase gene includes or consists of the nucleic acid sequence set forth as:

isoA
(SEQ ID NO: 12)
ATGACCGCAGCCAATGTGAAAATTCTGAATACCGAAGAAGTGCAGAATTT
TATTAATCTGCTGAGTGGTCTGGAACAAGAAGGTGGTAATCCGCGTATTA
AACAAATTATTCATCGTGTTGTGAGCGACCTGTTTAAAAGCATTGAGGAT
CTGGAAATTACCAGTGATGAATATTGGGCAGCCATTGCATATCTGAATCA
GCTGGGCACCAGCCATGAAGCAGGTCTGCTGAGTCCGGGTCTGGGTTTTG
ATCATTTTCTGGATATGCGTATGGATGCCATTGATGCAGCACTGGGTATT
GATAATCCGACACCGCGTACCATTGAAGGTCCGCTGTATGTTGCAGGCGC
ACCGGTTAGCCAGGGTTTTGCACGTATGGATGATGGTAGCGATCCGAATG
GTCATACCCTGATTCTGCATGGCACCATTTATAATGCAGATGGTCAGCCG
ATTCCGAATGCACAGGTTGAAATTTGGCATGCAAATACCAAAGGCTTTTA
TAGCCATTTTGATCCGACCGGTGAACAGACCCCGTTTAATATGCGTCGTA
CCATTATGACCGATGCACAGGGTCATTATCGTGTTCAGACCATTCTGCCG
AGCGGTTATGGTTGTCCGCCGAATGGTCCGACCCAGCAACTGCTGAATCA
GCTGGGTCGTCATGGTAATCGTCCGGCACATATTCATTTTTTTGTTAGCG
CAGATGGCTATCGTAAACTGACCACCCAGATTAATGTTGCGGGTGATCCG
TATACCTATGATGATTTTGCATTTGCAACCCGTGAAGGTCTGGTTGTTGA
AGCCATTGAACATACCGATCCGGCAACCAGCCAGCGTAATGGTGTTGAAG
GTCCGTTTGCAGAAATGGTTTTTGATCTGAAACTGAGCCGTCTGGTTGAT
GGTGTTGATAATCAGGTTGTTGATCGTCCGCGTCTGCAGGCATAA
isoB
(SEQ ID NO: 14)
AATCGCCAGCAGATTGATGCACTGGTTAAACAAATGAATGTGGATACCGC
AAAAGGTCCGGTTGATGAACGTATTCAGCAGGTTGTTGTTCGTCTGCTGG
GTGACCTGTTTCAGGCCATTGAGGATCTGGATATTCAGCCGAGCGAAGTT
TGGAAAGGTCTGGAATATCTGACCGATGCAGGTCAGGCAAATGAACTGGG
TCTGCTGGCAGCAGGTCTGGGTCTGGAACATTATCTGGATCTGCGTGCAG
ATGAAGCAGATGCAAAAGCAGGTATTACCGGTGGTACACCGCGTACCATT
GAAGGTCCGCTGTATGTTGCAGGCGCACCGGAAAGCGTTGGTTTTGCACG
TATGGATGATGGTAGCGAAAGCGATAAAGTTGATACCCTGATTATTGAAG
GCACCGTTACCGATACCGAAGGCAACATTATTGAAGGTGCCAAAGTTGAA
GTGTGGCATGCAAATAGCCTGGGTAATTATAGCTTTTTTGATAAAAGCCA
GAGCGATTTTAATCTGCGTCGTACCATTCTGACCGATGTGAATGGTAAAT
ATGTGGCACTGACCACCATGCCGGTTGGTTATGGTTGTCCGCCGGAAGGC
ACCACCCAGGCACTGCTGAATAAACTGGGTCGTCATGGTAATCGTCCGAG
CCATGTTCATTATTTTGTTAGCGCACCGGGTTATCGTAAACTGACCACCC
AGTTTAATATTGAAGGTGATGAATATCTGTGGGATGATTTTGCATTTGCA
ACCCGTGATGGTCTGGTTGCAACCGCAACCGATGTTACCGATGAAGCAGA
AATTGCCCGTCGTGAACTGGATAAACCGTTTAAACACATTACCTTTAATG
TGGAACTGGTGAAAGAAGCAGAAGCAGCACCGAGCAGCGAAGTTGAACGT
CGTCGTGCAAGCGCATAA

In some embodiments, a catechol 1,2-dioxygenase gene of use in the methods disclosed herein has a nucleic acid sequence at least 70%, 75%, 85%, 90%, 95%, 96%, 97%, 98% or 99% homologous to the nucleic acid sequence set forth in SEQ ID NOs: 12 and 14. Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein. Exemplary sequences can be obtained using computer programs that are readily available on the internet and the amino acid sequences set forth herein.

In some embodiments, the catechol 1,2-dioxygenase gene encodes a protein that includes or consists of the amino acid sequence set forth as:

IsoA
(SEQ ID NO: 13)
MTAANVKILNTEEVQNFINLLSGLEQEGGNPRIKQIIHRVVSDLFKSIED
LEITSDEYWAAIAYLNQLGTSHEAGLLSPGLGFDHFLDMRMDAIDAALGI
DNPTPRTIEGPLYVAGAPVSQGFARMDDGSDPNGHTLILHGTIYNADGQP
IPNAQVEIWHANTKGFYSHFDPTGEQTPFNMRRTIMTDAQGHYRVQTILP
SGYGCPPNGPTQQLLNQLGRHGNRPAHIHFFVSADGYRKLTTQINVAGDP
YTYDDFAFATREGLVVEAIEHTDPATSQRNGVEGPFAEMVFDLKLSRLVD
GVDNQVVDRPRLQA
IsoB
(SEQ ID NO: 15)
NRQQIDALVKQMNVDTAKGPVDERIQQVVVRLLGDLFQAIEDLDIQPSEV
WKGLEYLTDAGQANELGLLAAGLGLEHYLDLRADEADAKAGITGGTPRTI
EGPLYVAGAPESVGFARMDDGSESDKVDTLIIEGTVTDTEGNIIEGAKVE
VWHANSLGNYSFFDKSQSDFNLRRTILTDVNGKYVALTTMPVGYGCPPEG
TTQALLNKLGRHGNRPSHVHYFVSAPGYRKLTTQFNIEGDEYLWDDFAFA
TRDGLVATATDVTDEAEIARRELDKPFKHITFNVELVKEAEAAPSSEVER
RRASA

Similarly, the polypeptide encoded by the catechol 1,2-dioxygenase gene can have an amino acid sequence at least 80%, 85%, 90%, 95%, 96%, 97%, 98% or 99% homologous to the amino acid sequences set forth in SEQ ID NOs: 13 and 15.

In one example, the catechol 1,2-dioxygenase polypeptide retains a function of the wild-type protein, such as catalyzing conversion of catechol to cis,cis-muconate. Thus, a specific, non-limiting example of a catechol 1,2-dioxygenase polypeptide is a conservative variant of the catechol 1,2-dioxygenase polypeptide (such as a single conservative amino acid substitution, for example, one or more conservative amino acid substitutions, for example 1-10 conservative substitutions, 2-5 conservative substitutions, 4-9 conservative substitutions, such as 1, 2, 5 or 10 conservative substitutions). A table of conservative substitutions is provided above (Table 1).

D. 3,4-DHB dioxygenase

Some embodiments of the disclosed methods include expressing a heterologous 3,4-DHB dioxygenase gene in a prokaryotic cell, for example, in addition to expressing a heterologous asbF gene. Expression of an asbF gene and a 3,4-DHB dioxygenase gene in a prokaryotic cell results in production of β-cis,cis-muconic acid by the cell when it is cultured under conditions sufficient to produce β-cis,cis-muconic acid. 3,4-DHB dioxygenase is also known as protocatechuate 3,4-dioxygenase and is designated as EC 1.13.11.3. 3,4-DHB dioxygenase catalyzes the direct conversion of 3,4-DHB to β-cis,cis-muconic acid.

In some examples, the 3,4-DHB dioxygenase gene or protein is a bacterial 3,4-DHB dioxygenase gene or protein, for example, from Pseudomonas (such as P. putida, for example, P. putida F1 or P. putida KT2440, or P. marginata), Streptomyces (such as Streptomyces sp. strain 2065, Streptomyces sp. Strain D7, S. lividans, S, avermertilis, S. viridosporus, S. griseolus, S. setonii, or S. coelicolor), Agrobacterium (such as A. tumefaciens or A. radiobacter), Herbaspirillum (such as H. seropedicae), Rhodococcus (such as R. opacus), Burkholderia (such as B. cepacia), Azotobacter (such as A. vinelandii), Rhizobium (such as R. trifolii), Hydrogenophaga intermedia, Brevibacterium fuscum, or Acinetobacter (such as A. calcoaceticus, A. baylyi, or Acinetobacter sp. ADP1).

3,4-DHB dioxygenase has two subunits, an α subunit (expressed from a pcaG gene) and a β subunit (expressed from a pcaH gene). In some examples, the two subunits (α and β) form a heterodimer in solution to produce an active enzyme. In other examples, a homodimer of α subunits or a homodimer of β subunits produces an active enzyme. As utilized herein, a “3,4-DHB dioxygenase gene or protein” includes two subunits required for 3,4-DHB dioxygenase activity.

Nucleic acid and amino acid sequences for 3,4-DHB dioxygenase subunits are publicly available. For example, GenBank Accession Nos. AF109386 (nucleotides 3456-4061), NC_{—}009512 (nucleotides 5043283-5043888), NC_{—}002947 (nucleotides 5281003-5281608), NC_{—}005966 (nucleotides 1716541-1717170), NC_{—}003063 (nucleotides 1685833-1686453), AF312376 (nucleotides 728-1315), L14836 (nucleotides 980-1585), L05770 (nucleotides 22693-23322), and ATU32867 (nucleotides 1149-1769) disclose exemplary 3,4-DHB dioxygenase α subunit nucleic acid sequences and GenBank Accession Nos. AAD05270, YP_{—}001269821, NP_{—}746764, YP_{—}046376, NP_{—}356119, AAK84298, AAB41025, AAC37154, AAF34267 disclose exemplary 3,4-DHB dioxygenase α subunit amino acid sequences. GenBank Accession Nos. AF109386 (nucleotides 2676-3449), NC_{—}009512 (nucleotides 5043899-5044618), NC_{—}002947 (nucleotides 5281619-5282338), NC_{—}005966 (nucleotides 1715798-1716523), NC_{—}003063 (nucleotides 1686456-1687196), AF312376 (nucleotides 1-726), L14836 (nucleotides 250-969), L05770 (nucleotides 21950-22675), and ATU32867 (nucleotides 1772-2512) disclose exemplary 3,4-DHB dioxygenase β subunit nucleic acid sequences and GenBank Accession Nos. AAD05269, YP_{—}001269822, NP_{—}746765, YP_{—}046375, NP_{—}356118, AAK84297, AAB41024, AAC37153, and AAF34268 disclose exemplary 3,4-DHB dioxygenase β subunit amino acid sequences. Each of these sequences is incorporated by reference as provided by GenBank on Jan. 31, 2011. Additional 3,4-DHB dioxygenases are known in the art (see, e.g., Brown et al., Ann. Rev. Microbiol. 58:555-585, 2004; Davis et al., Inorg. Chem. 38:3676-3683, 1999).

In a particular example, the 3,4-DHB decarboxylase gene is from Streptomyces sp. Strain 2065, for example, the PcaHG genes (such as GenBank Accession No. AF109386 (nucleotides 2676-4061)). In some examples, the 3,4-DHB dioxygenase gene includes or consists of the nucleic acid sequences set forth as:

α-subunit (pcaG)
(SEQ ID NO: 16)
ATGACGACCATCGACACGAGCCGCCCGGAGTCCGTGCAGCCGACCCCGTC
GCACACGGTCGGCCCCTTCTACGGCTACGCGCTGCCCTTCCCCGGCGGCG
GCGACATCGCCCCGGTCGGCCACCCCGACACGATCACCGTCCAGGGCTAC
ATCTACGACGGCGAAGGCAAACCACTCCCCGACGCCTTCGTGGAACTCTG
GGGCCCCGACCCCGAGGGCAACCTCTCCACGACCGACGGCTCGATCCGGC
GCGACCCGGCCAGCGGCGGCTATCTCGGCCGCAACGGCGTGGAGTTCACC
GGCTGGGGCCGCATCCAGACGGACGCCAACGGCCACTGGTACGCACGGAC
GCTGCGCCCGGGAGCGCGCGGCCAAAGCGCCCCGTACCTGAGCGCGTGCG
TCTTCGCGCGCGGACTGCTGGTGCACCTCTTCACCCGCATCTACCTCCCG
GGCGACGAGCCCACGCTCACCGCGGACCCGCTGCTGTCCGGGCTCGACCC
GGCGCGGCGCGGCACGCTGATCGCGCGGGACGAGGGCAGGGGCACATACC
GTTTCGACATCCGCCTTCAGGGCGAAGGCGAGACGGTATTCCTGGAGTTC
CAGTGA
β-subunit (pcaH)
(SEQ ID NO: 18)
ATGACTCTCACCCAGCACGACATCGACCTCGAAATAGCGGCCGAGCACGC
GACGTACGAGAAGCGGGTCGCCGACGGCGCGCCGGTCGAGCACCACCCGC
GCCGCGACTACGCCCCGTACCGCTCCTCCACGCTCCGCCACCCGAAACAG
CCGCCGGTCACCATCGACGTCTCCAAGGACCCCGAACTGGTGGAGCTGGC
CTCGCCCGCGTTCGGCGAGCGGGACATCACGGAGATCGACAACGACCTGA
CCCGGCAGCACAACGGCGAGCCGATCGGGGAGCGGATCACCGTCTCCGGA
CGGCTGTTGGACCGTGACGGGCGCCCGATCCGCGGCCAGCTGGTCGAGAT
CTGGCAGGCGAACTCGGCCGGCCGCTACGCCCACCAGCGCGAGCAGCACG
ACGCCCCGCTGGACCCCAACTTCACTGGTGTGGGCCGCACGTTGACCGAC
GACGAGGGCGGGTACCACTTCACGACCGTCCAGCCGGGCCCCTACCCCTG
GCGCAACCACGTCAACGCCTGGCGCCCGGCGCACATCCACTTCTCGATGT
TCGGCTCGGCGTTCACGCAACGGCTCGTCACGCAGATGTACTTCCCGAGC
GACCCGCTGTTCCCGTACGACCCGATCATCCAGTCGGTGACGGACGACGC
GGCCCGCCAACGGCTCGTCGCGACGTACGACCACAGCCTGTCGGTGCCCG
AGTTCTCGATGGGCTACCACTGGGACATCGTGCTCGACGGCCCGCACGCC
ACCTGGATCGAAGAAGGACGCTGA

In some embodiments, a 3,4-DHB dioxygenase gene of use in the methods disclosed herein has a nucleic acid sequence at least 70%, 75%, 85%, 90%, 95%, 96%, 97%, 98% or 99% homologous to the nucleic acid sequences set forth in SEQ ID NOs: 16 and 18. Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein. Exemplary sequences can be obtained using computer programs that are readily available on the internet and the amino acid sequences set forth herein.

In some embodiments, the 3,4-DHB dioxygenase gene (pcaGH) encode two proteins that include or consist of the amino acid sequences set forth as:

α-subunit (pcaG)
(SEQ ID NO: 17)
MTTIDTSRPESVQPTPSHTVGPFYGYALPFPGGGDIAPVGHPDTITVQGY
IYDGEGKPLPDAFVELWGPDPEGNLSTTDGSIRRDPASGGYLGRNGVEFT
GWGRIQTDANGHWYARTLRPGARGQSAPYLSACVFARGLLVHLFTRIYLP
GDEPTLTADPLLSGLDPARRGTLIARDEGRGTYRFDIRLQGEGETVFLEF
Q
β-subunit (pcaH)
(SEQ ID NO: 19)
MTLTQHDIDLEIAAEHATYEKRVADGAPVEHHPRRDYAPYRSSTLRHPKQ
PPVTIDVSKDPELVELASPAFGERDITEIDNDLTRQHNGEPIGERITVSG
RLLDRDGRPIRGQLVEIWQANSAGRYAHQREQHDAPLDPNFTGVGRTLTD
DEGGYHFTTVQPGPYPWRNHVNAWRPAHIHFSMFGSAFTQRLVTQMYFPS
DPLFPYDPIIQSVTDDAARQRLVATYDHSLSVPEFSMGYHWDIVLDGPHA
TWIEEGR

Similarly, the polypeptide encoded by the 3,4-DHB dioxygenase gene can have amino acid sequences at least 80%, 85%, 90%, 95%, 96%, 97%, 98% or 99% homologous to the amino acid sequences set forth in SEQ ID NOs: 17 and 19.

In one example, the 3,4-DHB dioxygenase polypeptide retains a function of the wild-type protein, such as catalyzing conversion of 3,4-DHB to β-cis,cis-muconic acid. Thus, a specific, non-limiting example of a 3,4-DHB dioxygenase polypeptide is a conservative variant of the 3,4-DHB dioxygenase polypeptides (such as a single conservative amino acid substitution, for example, one or more conservative amino acid substitutions, for example 1-10 conservative substitutions, 2-5 conservative substitutions, 4-9 conservative substitutions, such as 1, 2, 5 or 10 conservative substitutions in one or both subunits). A table of conservative substitutions is provided above (Table 1).

### IV. Expression of Heterologous Genes in Prokaryotic Cells

The disclosed methods include expression of one or more heterologous genes (such as an asbF gene, a 3,4-DHB decarboxylase gene, a catechol 1,2-dioxygenase gene, or a 3,4-DHB dioxygenase gene) in a prokaryotic cell (such as a bacterium or a cyanobacterium). Methods of expressing heterologous genes in a prokaryotic cell are well known to one of skill of the art. For example, a heterologous gene is included in a suitable bacterial or cyanobacterial expression vector. Non-limiting examples of suitable host cells include bacteria and archaea. Exemplary, non-limiting, methods are described below.

In some examples, the heterologous gene is codon-optimized for the cell in which it is to be expressed. Codon usage bias, the use of synonymous codons at unequal frequencies, is ubiquitous among genetic systems (Ikemura, J. Mol. Biol. 146:1-21, 1981; Ikemura, J. Mol. Biol. 158:573-97, 1982). The strength and direction of codon usage bias is related to genomic G+C content and the relative abundance of different isoaccepting tRNAs (Akashi, Curr. Opin. Genet. Dev. 11:660-6, 2001; Duret, Curr. Opin. Genet. Dev. 12:640-9, 2002; Osawa et al., Microbiol. Rev. 56:229-64, 1992). Codon usage can affect the efficiency of gene expression. For example, in Escherichia coli (Ikemura, J. Mol. Biol. 146:1-21, 1981; Xia Genetics 149:37-44, 1998) the most highly expressed genes use codons matched to the most abundant tRNAs (Akashi and Eyre-Walker, Curr. Opin. Genet. Dev. 8:688-93, 1998).

Codon-optimization refers to replacement of a codon in a nucleic acid sequence with a synonymous codon (one that codes for the same amino acid) more frequently used (preferred) in the organism. Each organism has a particular codon usage bias for each amino acid, which can be determined from publicly available codon usage tables (for example see Nakamura et al., Nucleic Acids Res. 28:292, 2000 and references cited therein). For example, a codon usage database is available on the world wide web at kazusa.or.jp/codon. One of skill in the art can modify a nucleic acid encoding a particular amino acid sequence, such that it encodes the same amino acid sequence, while being optimized for expression in a particular cell type (such as a bacterial or cyanobacterial cell). In one particular example, the asbF nucleic acid sequence of SEQ ID NO: 1 is suitable for expression in bacteria (such as E. coli), while the asbF nucleic acid sequence of SEQ ID NO: 2 is suitable for expression in cyanobacteria (such as Synechocystis). However, one of skill in the art will recognize that a nucleic acid does not have to be optimized for expression in a particular organism in order to be used for gene expression in the selected organism.

The choice of the expression system will be influenced by the features desired for the expressed polypeptides. Any transducible cloning vector can be used as a cloning vector for the nucleic acid constructs presently disclosed. If large clusters are to be expressed, it is preferable that phagemids, cosmids, P1s, bacterial artificial chromosomes (BACs), P1 artificial chromosomes (PACs), or similar cloning vectors are used for cloning the nucleotide sequences into the host cell and subsequent expression. These vectors are advantageous due to their ability to insert and stably propagate larger fragments of DNA, compared to M13 phage and lambda phage.

In an embodiment, one or more of the disclosed heterologous genes and/or variants thereof can be inserted into one or more expression vectors, using methods known to those of skill in the art. Vectors are used to introduce genes or a gene cluster into bacterial cells may be either integrated or episomal. Vectors include one or more expression cassette including expression control sequences operably linked to the desired heterologous nucleic acid. However, the choice of an expression cassette may depend upon the host system selected and features desired for the expressed polypeptide or natural product. An expression cassette includes nucleic acid elements that permit expression of a gene in a host cell. Typically, the expression cassette includes a promoter that is functional in the selected host system that is operably linked to the gene to be expressed. The promoter can be constitutive or inducible. In an embodiment, the expression cassette includes for each heterologous nucleic acid a promoter, ribosome binding site, a start codon (ATG) if necessary, and optionally a region encoding a leader peptide in addition to the desired DNA molecule and stop codon. In addition, a 3′ terminal region (translation and/or transcription terminator) can be included within the cassette. The heterologous nucleic acid constituted in the DNA molecule may be solely controlled by the promoter so that transcription and translation occur in the host cell. Promoter encoding regions are well known and available to those of skill in the art. Examples of promoters include bacterial or cyanobacterial promoters (such as those derived from sugar metabolizing enzymes, such as galactose, lactose and maltose), promoter sequences derived from biosynthetic enzymes such as tryptophan, the beta-lactamase promoter system, bacteriophage lambda PL and TF and viral promoters. Additional promoters include light inducible promoters, such as PsbAII (see, e.g., U.S. Pat. Publ. No. 2009/0155871; incorporated herein by reference). In another example, a promoter is a T7 promoter.

The presence of additional regulatory sequences within the expression cassette may be desirable to allow for regulation of expression of the one or more heterologous genes relative to the growth of the host cell. These regulatory sequences are well known in the art. Examples of regulatory sequences include sequences that turn gene expression on or off in response to chemical or physical stimulus as well as enhancer sequences. In addition, to the regulatory sequences, selectable markers can be included to assist in selection of transformed cells. For example, genes that confer antibiotic resistance or sensitivity to the plasmid may be used as selectable markers.

It is contemplated that one or more of the heterologous genes of interest can be cloned into one or more recombinant vectors as individual cassettes, with separate control elements, or under the control of a single control element (e.g., a promoter). In an embodiment, the cassettes include two or more restriction sites to allow for the easy deletion and insertion of one or more open reading frames so that hybrid synthetic pathways can be generated. The design and use of such restriction sites is well known in the art and can be carried out by using techniques described above such as PCR or site-directed mutagenesis. Proteins expressed by the transformed cells can be recovered according to standard methods well known to those of skill in the art. For example, proteins can be expressed with a convenient tag to facilitate isolation (such as a 6×His tag). Further, the resulting polypeptide can be purified by affinity chromatography by using a ligand (such as an antibody) that binds to the polypeptide.

A. Expression in Bacteria

The disclosed methods include expression of a heterologous gene (such as a DHS dehydratase gene (for example, asbF), a 3,4-DHB decarboxylase gene, a 3,4-DHB-dioxeygenase gene, and/or a catechol 1,2-dioxygenase gene) is in bacteria. Bacterial cells are available from numerous sources, including commercial sources known to those skilled in the art, such as the American Type Culture Collection (ATCC; Manassas, Va.). Commercial sources of cells used for recombinant protein expression also provide instructions for usage of such cells. Suitable bacteria for use in the methods disclosed herein include but are not limited to Escherichia coli, Bacillus (for example, B. thuringiensis, B. cereus, and B. anthracis), and Streptomyces sp. In one specific example, the bacterium is E. coli. Bacterial cells are available commercially, for example from American Type Culture Collection (Manassas, Va.).

In an embodiment, a recombinant expression system is selected from bacterial hosts. One representative heterologous host system for expression of one or more of the disclosed heterologous genes is E. coli. E. coli is an attractive artificial expression system because it is fast growing and easy to genetically manipulate. In some examples, the heterologous gene is placed under control of a promoter (such as an inducible promoter, for example, an inducible T7 promoter). In additional examples, the promoter is an arabinose-inducible promoter (for example, the pBAD system), a lac promoter (direct IPTG/lactose induction), a trc promoter (direct IPTG/lactose induction), a tetracycline-inducible promoter, or a pho promoter (phosphate deprivation induced).

Recent advances in E. coli-based expression systems have greatly aided efforts to simultaneously express multiple genes in a single host organism. Multiple ORFs from a complex biosynthetic system can now be expressed simultaneously in E. coli. To ensure adequate and coordinate production of multiple biosynthetic enzymes from a single pathway, each nucleic acid encoding a heterologous gene is optionally placed under control of a single type of promoter, such as the inducible T7 promoter. Novagen (San Diego, Calif.) has introduced the Duet™ vectors, which are designed with compatible replicons and drug resistance genes for effective propagation and maintenance of four plasmids in a single cell. This allows for the coexpression of up to eight different proteins. In other examples, the vector is a pET vector, such as a pET21 or pET28 vector. pET and pET-based vectors are commercially available, for example from Novagen (San Diego, Calif.), or Clontech (Mountain View, Calif.).

In one example, the vector is pET21a or pET28a. In some examples, the pET vector includes a resistance marker (e.g. ampicillin or kanamycin resistance) and a T7 promoter. The multiple cloning site has been manipulated such that more than one gene (such as 2, 3, 4, 5, 6, 7, 8, 9, 10, 11, 12 13, 14 or more) can be expressed from a single vector. In one example, up to at least 13 genes are expressed from a single plasmid (e.g., Watanabe et al., Nat. Chem. Biol. 2:423-428, 2006; incorporated herein by reference). In some examples, the genes are expressed as a multicistronic product (for example, a bi-cistronic, tri-cistronic, etc. product), with a single mRNA and multiple polypeptides produced. In other examples, the genes are expressed as multiple monocistronic products, with an individual mRNA and polypeptide produced for each gene.

In some examples, the plasmid introduced is extrachromosomally and replicated within the host. In other examples, after introduction of the plasmid, a double homologous recombination event occurs and the one or more genes are inserted into the genome.

Another representative heterologous host system for expression of one or more of the disclosed heterologous genes is Bacillus sp. Bacillus sp. are useful heterologous host systems because they are easily grown and plasmids and cosmids for the expression and/or integration of biosynthetic gene clusters are well characterized (e.g., Brockmeier et al., Curr. Microbiol, 52:143-148, 2006. Vectors for expression of heterologous genes in Bacillus sp. are commercially available, for example from MoBiTec (Gottingen, Germany) and Takara Bio Inc. (Madison, Wis.). In one example, the plasmid is pHY300PLK or pTZ12.

A further representative heterologous host system for expression of one or more of the disclosed heterologous genes is Streptomyces sp. Streptomyces sp. are useful heterologous host systems because they are easily grown, plasmids and cosmids for the expression and/or integration of biosynthetic gene clusters are well characterized, and they house many of the modifying and auxiliary enzymes required to produce functional pathways (Donadio et al., J. Biotechnol., 99:187-198, 2002).

Transformation of a bacterial cell with recombinant DNA can be carried out by conventional techniques as are well known to those skilled in the art. Where the host is bacterial, such as, but not limited to, E. coli, competent cells which are capable of DNA uptake can be prepared from cells harvested after exponential growth phase and subsequently treated by the CaCl₂ method using procedures well known in the art. Alternatively, MgCl₂ or RbCl can be used. Bacteria can also be transformed by electroporation, conjugation, or transduction.

B. Expression in Cyanobacteria

In some examples, a heterologous gene (such as a DHS dehydratase gene (for example, asbF), a 3,4-DHB decarboxylase gene, a 3,4-DHB-dioxeygenase gene, and/or a catechol 1,2-dioxygenase gene) is included in a vector (such as a plasmid) for expression in cyanobacteria. Cyanobacterial cells are available commercially, for example from American Type Culture Collection (Manassas, Va.). Suitable cyanobacteria for use in the methods disclosed herein include but are not limited to Synechocystis sp. (for example, Synechocystis PCC6803 or Synechocystis PCC9714), Synechococcus sp., Spirulina sp., Anabaena sp. (for example, Anabaena variabilis), Trichodesmium, Crocosphaera, and Arthrospira maxima. In one specific example, the cyanobacterium is Synechocystis PCC6803. One of skill in the art can identify recombinant expression systems suitable for expression of a heterologous gene in cyanobacteria. See, e.g., U.S. Pat. Publ. Nos. 2009/0104656 and 2009/0155871; incorporated herein by reference. One of skill in the art can select a suitable vector for expression of a heterologous gene in a particular cyanobacterial species. In some examples, T7Blue-T (Novagen, Madison, Wis.), pUC19 (New England Biolabs, Ipswich, Mass.), pBluescript (Stratagene, La Jolla, Calif.), and pGEM-T (Promega, Madison, Wis.) vectors can be used to transform cyanobacteria.

In one example, a vector for a double homologous recombination event into Synechocystis (such as Synechocystis PCC6803) is provided herein (SEQ ID NO: 20). This plasmid (pATK_muc_v1) harbors the genes encoding particular AsbF, 3,4-DHB decarboxylase, and catechol 1,2-dioxygenase proteins. However, one of skill in the art can modify the plasmid to include sequences that encode other AsbF, 3,4-DHB decarboxylase, and/or catechol 1,2-dioxygenase proteins, or other proteins, such as 3,4-DHB dioxygenase. In some examples, the plasmid includes sequences encoding one, two, three, or all of AsbF, 3,4-DHB decarboxylase, catechol 1,2-dioxygenase and 3,4-DHB dioxygenase.

In other examples, a representative vector construct for expression of cyanobacterial genes is pRL489, which contains the cyanobacterial replicon (pDU1), an E. coli replicon (oriV), an origin of transfer (oriT) for intergenic conjugation, the neomycin resistance gene (npt) conferring resistance to neomycin and kanamycin (see, e.g., Billi et al., J. Bacteriol., 183(7):2298-2305, 2001). For heterologous expression of a heterologous gene in cyanobacteria, a strong constitutive cyanobacterial promoter (such as PpsbA derived from chloroplast of Amaranthus hybridus) or an inducible promoter (such as a light inducible promoter PsbAII) is placed upstream of a multiple cloning site (MCS). In addition, four cyanobacterial plasmid constructs (pRL271, pRL528, pRK2013, and pSCR202) are available (for example, from American Type Culture Collection, Manassas, Va.; see also, GenBank Accession Nos. L05081 and AY622813). pRK2013 is a mobilizer plasmid and pRL528 is a helper plasmid required for triparental conjugation. pRL271 is a conjugatable, non-replicating cyanobacterial vector that can be used in triparental conjugation experiments for the integration of biosynthetic genes into the host chromosome or for knockout studies. pSCR202 is a multicopy replicating plasmid that can be transformed into cyanobacteria using electroporation.

In some examples, the plasmid introduces the one or more genes of interest into the cyanobacterial chromosome through a double homologous recombination event. In one non-limiting example, a plasmid is constructed to contain about a 500 bp homologous region, both upstream and downstream of the gene(s) of interest, to the psbAII gene that encodes for highly expressed D1 protein of photosystem II. In addition, the psbAII strong promoter is embedded upstream of the gene(s) of interest. Following methods established by Vermaas et al. (J. Appl. Phycol. 8:263-273, 1996) for both a positive (kanamycin resistance) and negative selection (sucrose resistance) the cyanobacterial vector can move the gene(s) of interest into the identical site where the sacB/aphX genes were inserted into the psbAII region of the genome.

Transformation of a cyanobacterial cell with recombinant DNA can be carried out by conventional techniques as are well known to those skilled in the art. Where the host is cyanobacterial, such as, but not limited to, Synechocystis, cells can be transformed by passive uptake of DNA and homologous recombination (see, e.g., Kufryk et al., FEMS Microbiol. Lett. 206:215-219, 2002). Cyanobacteria can also be transformed by electroporation (e.g., Thiel and Poo, J. Bacteriol. 171:5743-5746, 1989) or conjugation (e.g., Elhai and Wolk, Meth. Enzymol. 167:747-754, 1988).

C. Selection of Cells with High Growth Rate

Expression of one or more heterologous genes in a cell can place metabolic stress on the cell and reduce cell growth and/or viability. In some embodiments disclosed herein, cells having one or more desired characteristics (such as high growth rate) are identified and selected. In some examples, the methods include identifying and selecting cells with increased auto-fluorescence, cell size, or optical density, for example, compared to a control. In some examples, the cells express one or more heterologous genes (such as asbF, 3,4-DHB decarboxylase, 3,4-DHB dioxygenase, or catechol 1,2-dioxygenase), while in other examples, the cells do not express a heterologous gene.

In one embodiment, cells (such as Synechocystis PCC6803 cells) are cultured using routine methods (for example, culture in media on a rotary shaker). Auto-fluorescence of the cells is measured at specified time intervals, such as every hour (for example, every 2 hours, 4 hours, 8 hours, 12 hours, 18 hours, 24 hours, 36 hours, 48 hours, or more). Methods of measuring auto-fluorescence of cells, such as utilizing a flow cytometer, are routine and well known to one of skill in the art. In some examples, cells with a selected level of auto-fluorescence (for example, cells in the highest third of fluorescent cells in a culture) are sorted utilizing a flow cytometer and utilized in the methods for production of commodity compounds disclosed herein. In some examples, a population of cells with fluorescence of at least about 2-fold higher than the remainder of the cells (such as about 2-fold, 3-fold, 4-fold, 5-fold, or even higher) is selected.

### V. Purification of Compounds of Interest In some embodiments of the methods disclosed herein, one or more compounds produced by expression of a heterologous asbF gene (alone or in combination with expression of one or more additional heterologous genes) are purified from the cells. Methods for purifying such compounds (such as 3,4-DHB, catechol, cis,cis-muconate, adipic acid, β-cis,cis-muconic acid, or β-carboxyadipic acid) are well known to one of skill in the art.

Methods to purify 3,4-DHB from aqueous media have previously been developed for diverse bacteria (Koppisch et al., Biometals 21:581-589, 2008). 3,4-DHB can be readily quantified in solution via UV or NMR methods, and extracted from aqueous systems either via organic solvents or by binding and elution from Dowex-type resins (Li et al., J. Am. Chem. Soc. 127:2874-2882, 2005). Methods for continuous extraction of 3,4-DHB from E. coli fermenters have been described and can be readily applied to other bacteria or cyanobacteria. 3,4-DHB accumulation can be quantitated in media as well as intracellular levels. In the event that 3,4-DHB is not efficiently exported from the cells, coexpression of AsbF with catecholate siderophore efflux proteins may be utilized (Grass, Biometals 19:159-172, 2006; Bleuel et al., J. Bacteriol. 187:6701-6707, 2005). The role of these proteins in E. coli is to bind and transport catecholate siderophores from the cytosol into the environment, a function that is likely extended to the 3,4-DHB monomers as well.

In some examples, 3,4-DHB is purified from cell culture by organic extraction (for example, extraction of cell culture supernatant with an organic solvent, such as ethyl acetate). Other organic solvents also suitable for extraction include chloroform, methylene chloride, variable-length halogenated alkanes, variable-length carbon based alcohols, esters, ethers, furans, and any other organic solvent where a biphasic mixture is generated and 3,4-DHB has preference for the organic layer versus the aqueous solution from which it was derived. The 3,4-DHB can be isolated through either continuous (Soxhlet extractor) or discontinuous (separatory funnel) extraction methods. Following one or more optional washing steps, the organic solvent is evaporated, resulting in purified 3,4-DHB. In other examples, 3,4-DHB is purified from cell culture with an anion-exchange resin (such as AMBERLITE® XAD2), for example, by batch or continuous binding the culture with the resin. Additional resins that can be used include AMBERLITE® XAD-4, SUPELPAK®-2, or SUPELPAK®-4 resins. Boronate affinity chromatography (Barnes and Ishimaru, Biometals 12:83-87, 1999) can also be used to purify 3,4-DHB. The resin can be washed (for example, to remove cells or media) and the product can be eluted with a polar organic solvent (such as an alcohol, ester, or related halogenated organic solvent, for example, methanol) to produce purified 3,4-DHB.

Similarly, methods of purifying catechol (for example, from bacterial cell culture) are well known in the art. In some examples, catechol is purified by boronate affinity chromatography (e.g., Barnes and Ishimaru, Biometals 12:83-87, 1999) or by extraction and distillation (e.g., U.S. Pat. No. 5,679,223).

In some examples, catechol is purified from cell culture by organic extraction (for example, extraction of supernatant with an organic solvent, such as ethyl acetate).

Other organic solvents also suitable for extraction include chloroform, methylene chloride, variable-length halogenated alkanes, variable-length carbon based alcohols, esters, ethers, furans, and any other organic solvent where a biphasic mixture is generated and catechol has preference for the organic layer versus the aqueous solution from which it was derived. Catechol can be isolated through either continuous (Soxhlet extractor) or discontinuous (separatory funnel) extraction methods. Following one or more optional washing steps, the organic solvent is evaporated, resulting in purified catechol. In other examples, catechol is purified from cell culture with an anion-exchange resin (such as AMBERLITE® XAD2), for example, by batch or continuous binding the culture with the resin. Additional resins can include AMBERLITE® XAD-4, SUPELPAK®-2, or SUPELPAK®-4 resins. Boronate affinity chromatography (Barnes and Ishimaru, Biometals 12:83-87, 1999) can also be used to purify catechol. The resin can be washed (for example, to remove cells or media) and the product can be eluted with a polar organic solvent (such as an alcohol, ester, or related halogenated organic solvent, for example, methanol) to produce purified catechol.

Similarly, methods of purifying and/or producing cis,cis-muconate and adipic acid (for example, from bacterial cell culture) are well know in the art. In some examples, the methods are as described above for purifying 3,4-DHB and catechol (e.g., organic extraction or resin binding). However, for purifying cis,cis-muconate, the supernatant from a cell culture is acidified to a pH of about 4 or less prior to organic extraction.

Adipic acid is produced from cis,cis-muconic acid by reduction of the double bonds. In some examples, adipic acid is produced by passing hydrogen gas through the solvent containing cis,cis-muconic acid in the presence of a suitable catalyst (such as platinum, palladium, rhodium, ruthenium, Raney nickel, or a combination of two or more thereof). In some examples, pressure is added to the system in order to accelerate the hydrogenation process. The catalyst can be recovered by filtration and regenerated. The resulting product, adipic acid, is recovered using one or more of the following methods: recrystallization, resins, or column chromatography (for example, as described above).

Similarly, methods of producing β-carboxy cis,cis-muconate and β-carboxy adipic acid (for example, from bacterial cell culture) are well known in the art. Purification of β-carboxy cis,cis-muconate can be accomplished using the methods described for cis,cis-muconate. Similarly, β-carboxy cis,cis-muconate is converted to β-carboxyadipic acid by hydrogenation of the double bonds, for example, as described above for adipic acid.

The following examples are provided to illustrate certain particular features and/or embodiments. These examples should not be construed to limit the invention to the particular features or embodiments described.

### EXAMPLES

### Example 1

### Production of 3,4-Dihydroxybenzoate in E. coli

This example describes expression of a 3,4-DHB-producing enzyme in E. coli and production of 3,4-DHB in culture.

The asbF gene of Bacillus thuringiensis 97-27 encodes a unique enzyme that catalyzes conversion of 3-dehydroshikimate (DHS) to 3,4-DHB (Fox et al., Biochemistry 47:12251-12253, 2008). asbF (SEQ ID NO: 1) was cloned into the pET28a(+) vector at the NdeI/EcoRI restriction sites and transformed into E. coli BL21(DE3) by the heat shock method. The transformed host was subsequently grown at 37° C. in LB media containing 50 μg/ml kanamycin until an OD₆₀₀ of about 0.4 was reached. The culture was then inoculated into fresh LB and asbF synthesis was induced with 0.4-1.0 μM isopropyl β-D-1-thiogalactopyranoside (IPTG) when the culture reached an OD₆₀₀ of 0.5. The induced cells were grown overnight at 30° C. The culture was centrifuged, and the cells were resuspended in M9 media supplemented with salts (magnesium, calcium, manganese, sodium, potassium, and variations thereof) and a range of glucose concentrations (1-100 mM). Production and release of 3,4-DHB by the cells was monitored by UV spectroscopy, thin layer chromatography (TLC), NMR spectroscopy, and where appropriate, mass spectrometry. The 3,4-DHB was extracted and isolated using ethyl acetate or DOWEX AG1 ion exchange resin.

Product formation was quantified as a function of time by monitoring the increase in absorbance at 290 nm as measured by UV spectroscopy. For example, after 1 hour of bacterial growth in M9 media with salt supplementation and glucose, a small amount (˜1 mL) was removed from the shake flask and acidified to pH 2 with either acetic or hydrochloric acid. The sample was extracted directly into a polar organic solvent (e.g. ethyl acetate) followed by solvent evaporation and resuspension in water. Quantification of crude product formation was by both gravimetric and UV spectroscopic methods. Assessment of purity was determined by both ¹H and ¹³C NMR spectroscopy.

The induced cells produced a large amount of AsbF protein, which was more than 50% of the total protein content of the cells. This was determined by direct visualization or densitometry.

Furthermore, the conversion of glucose to 3,4-DHB approached rates of about 125 μM/hour. Various growth and product isolation conditions produced approximately 5-40% crude yield of 3,4-DHB from glucose-based available carbon to 3-DHS (43% mol/mol). The conditions and yields were as follows:

Condition 1: A 500 mL culture of previously overexpressed AsbF in E. coli was resuspended in the identical volume of M9 salts with 60 mM glucose and incubated at 30° C. overnight with shaking (200 rpm). Product isolation and characterization by extraction into ethyl acetate resulted in an approximate 3,4-DHB yield of 10 mM (˜40%).

Condition 2: A 500 mL culture of previously overexpressed AsbF in E. coli was resuspended in the identical volume of M9 salts with 60 mM glucose plus 0.2% casamino acids and incubated at 30° C. overnight with shaking (200 rpm). Product isolation and characterization by extraction into ethyl acetate resulted in an approximate 3,4-DHB yield of ˜5 mM (˜20%).

Condition 3: A 500 mL culture of previously uninduced AsbF (no forced enzyme expression) in E. coli was resuspended in the identical volume of M9 salts with 60 mM glucose and incubated at 30° C. overnight with shaking (200 rpm). Product isolation and characterization by extraction into ethyl acetate resulted in an approximate 3,4-DHB yield of ˜3.5 mM (˜15%).

Condition 4: A 500 mL culture of previously uninduced AsbF (no forced enzyme expression) in E. coli was resuspended in the identical volume of M9 salts with 60 mM glucose and 0.2% casamino acids and incubated at 30° C. overnight with shaking (200 rpm). Product isolation and characterization by extraction into ethyl acetate resulted in an approximate 3,4-DHB yield of 2 mM (˜10%).

Condition 5: A 500 mL culture of previously overexpressed AsbF in E. coli was resuspended in 50 mL of M9 salts with 60 mM glucose and incubated at 30° C. overnight with shaking (200 rpm). Product isolation and characterization by extraction into ethyl acetate resulted in an approximate 3,4-DHB yield of <1 mM (<5%).

Condition 6: A resuspended 500 mL culture of previously overexpressed AsbF in E. coli was diluted 1/10 (50 mL of culture in 500 mL) in M9 salts with 60 mM glucose and incubated at 30° C. overnight with shaking (200 rpm). Product isolation and characterization by extraction into ethyl acetate resulted in an approximate 3,4-DHB yield of <1 mM (<5%).

### Example 2

### Production of Catechol in E. coli

This example describes expression of a 3,4-DHB-producing enzyme and a 3,4-DHB decarboxylase in E. coli and production of catechol in culture.

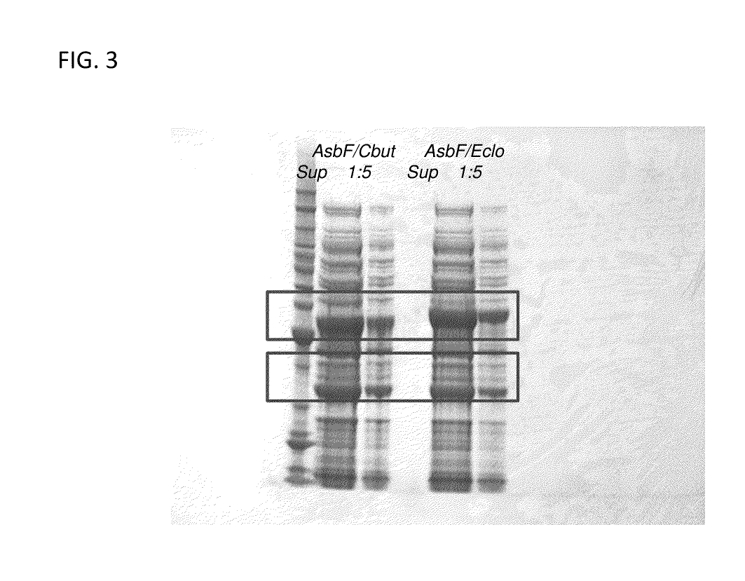

E. coli BL21 DE3 cells were transformed with equally proportional amounts of vectors containing asbF (vector including Kanamycin resistance gene, Example 1) and Enterobacter cloacae 3,4-DHB decarboxylase (vector including Ampicillin resistance gene). Positive transformants were isolated off of LB agar plates containing 50 μg/ml kanamycin and 100 μg/ml ampicillin and step-wise grown into 50 mL LB containing 50 μg/ml kanamycin and 100 μg/ml ampicillin cultures. The cultures were monitored using OD₆₀₀ and when the OD was at about 0.3-0.4, the culture was induced with IPTG. Initial inductions were done using 0.1 mM IPTG but this resulted in low expression of 3,4-DHB decarboxylase. Subsequent inductions done with 1 mM IPTG yielded better expression of 3,4-DHB decarboxylase. After induction, the cultures were incubated at 30° C. with shaking (200 rpm) over night (about 16 hours). The cultures were then moved into M9 media after two washes with 50 mL of M9 salts. Once in M9 media the cultures were allowed to incubate again at 30° C. with shaking (200 rpm) for various amounts of time before undergoing extraction procedures to isolate any secreted products. Expression of asbF and 3,4-DHB decarboxylase was confirmed by analyzing total protein extracts from the cells by gel electrophoresis (FIG. 3).

In some cases, catechol was isolated by organic extraction. M9 cultures were spun down and the supernatant removed for product isolation. The supernatant was acidified to a pH of 2, and then decanted into a separatory funnel where an equal volume of ethyl acetate was added, and mixed with shaking. After two rounds of shaking the supernatant was removed, and the organic layer was washed twice with equal volumes of brine. Finally the organic layer was moved into a beaker and further dried by the addition of MgSO₄. The organic layer was then removed by rotary evaporation, leaving the solid product which was reconstituted in deionized H₂O and analyzed by thin layer chromatography (TLC), UV spectroscopy, and NMR. The optimal concentration of catechol production reached was about 300 μM. Cells could be reconstituted in M9 media and continue catechol production.

In other cases, catechol was isolated by resin extraction. Once the induced cultures were moved to M9 media, 10 mL of AMBERLITE® XAD2 resin (Supelco, Sigma-Aldrich Catalog No. 10357) was added to the media and allowed to batch bind over night at 30° C. with shaking (200 rpm). Various quantities of XAD2 resin were added to the culture, and 10 mL of a 1 mg/ml suspension resulted in isolation of the maximum amount of catechol. After overnight batch binding, the resin was isolated by flowing the culture over a column and the cells and M9 passing through with no detectable loss of cells as determined by both optical density at 600 nm and by cell counting on agar plates. More resin was added and catechol production continued. Once the resin was isolated in the column it was washed with 3 bed volumes of deionized H₂O to removed any remaining cells or M9 media. Next the catechol was eluted using 3 bed volumes of methanol and the eluant was collected in a glass beaker. The collected methanol was then removed via rotary evaporation and the dry product was reconstituted in deionized H₂O and analyzed by TLC, UV spectroscopy, and ¹H NMR spectroscopy. The isolated amount of catechol indicated it was at a concentration of about 3.5 mM in the culture.

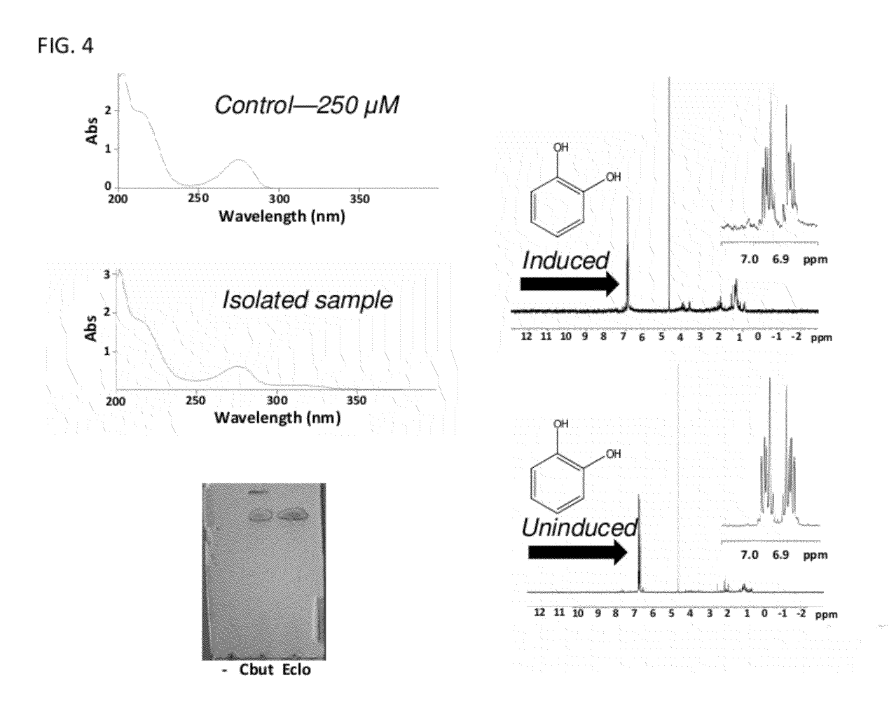

As shown in FIG. 4, catechol was formed and could be isolated from E. coli expressing asbF and 3,4-DHB decarboxylase, as assessed by UV-Vis spectroscopy, TLC, and ¹H NMR.

### Example 3

### Flow Cytometry Analysis of Synechocystis sp. PCC 6803 Growth

This example describes representative flow cytometry methods for measuring growth rates of cultures of Synechocystis.

### Materials and Methods:

PCC 6803 Growth Conditions: Synechocystis PCC 6803 cells were cultured in BG-11 media in a rotatory shaker at 25° C. under 25 μmol photons m⁻² s⁻¹ on a 16 hour light and 8 hour dark cycle, and with exposure to air through a mesh top. The cell growth was monitored using UV spectroscopy and measuring the absorbance of 1 mL of cells at 685 nm or 730 nm.

Flow cytometry analysis of PCC 6803 autofluorescence: The daily analysis of PCC 6803 auto-fluorescence was read by a FACSCalibur™ 1 flow cytometer (BD Biosciences, San Jose, Calif.). PCC 6803 cells were diluted to an OD₆₈₅ of 0.05 and analyzed at a flow rate of 12 μL/second. The auto-fluorescence was detected by excitation with a 488 nm laser and the emission detected at 650 nm. Analysis of the collected data was done using WinMDI version 2.9 software.

PCC 6803 Cell sorting: PCC 6803 cells were collected from a stably growing culture under conditions described previously. The cells were sorted using a FACSAria™ cell sorter (BD Biosciences) based on their fluorescence at 670 nm. The top one third (about 33%) of fluorescent cells was collected as accelerated growers (P3) and the lower two thirds were collected as slow growers (P4).

### Results

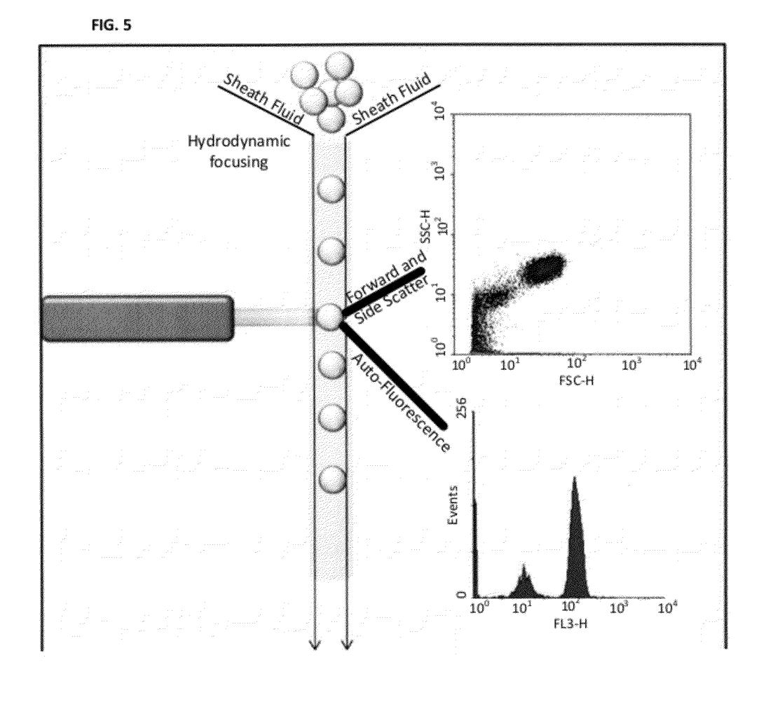

Identification of metabolic markers: In order to identify fast-growing strains of PCC 6803, growing cultures were monitored using flow cytometry as well as measuring the optical density at wavelength 685 nm (OD₆₈₅). OD₆₈₅ measures the cell density and can be used to determine growth rates of cultures over time. Flow cytometry provides forward and side scatter measurements, which report on cell size and complexity, as well as the relative intensity of emitted fluorescence. Flow cytometry is capable of providing these measurements by analyzing individual cells using lasers of specific wavelengths; the scattering of the laser provides size and complexity information, and the excitation of the PCC 6803 photosystem pigments result in measurable auto-fluorescence (FIG. 5).

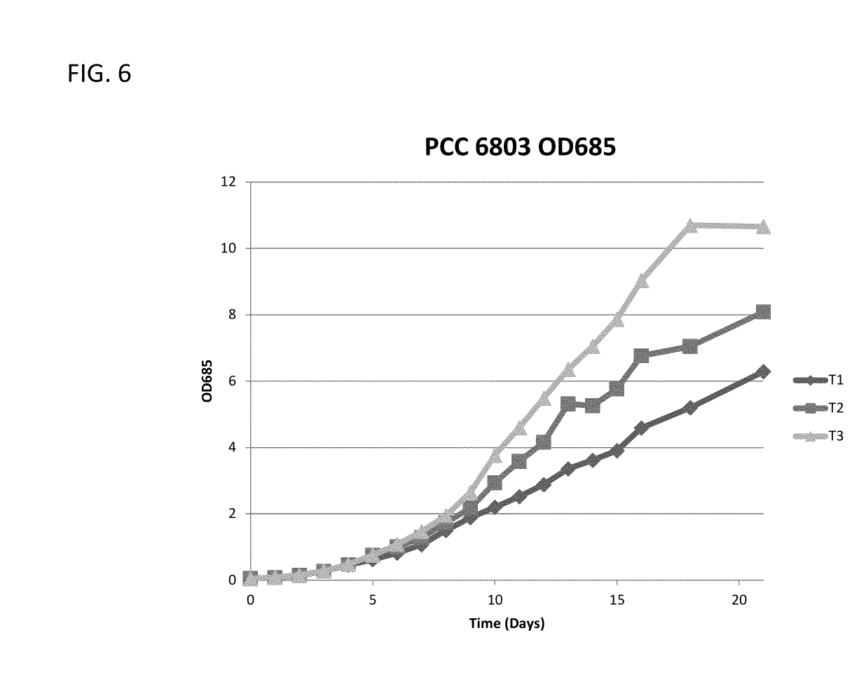

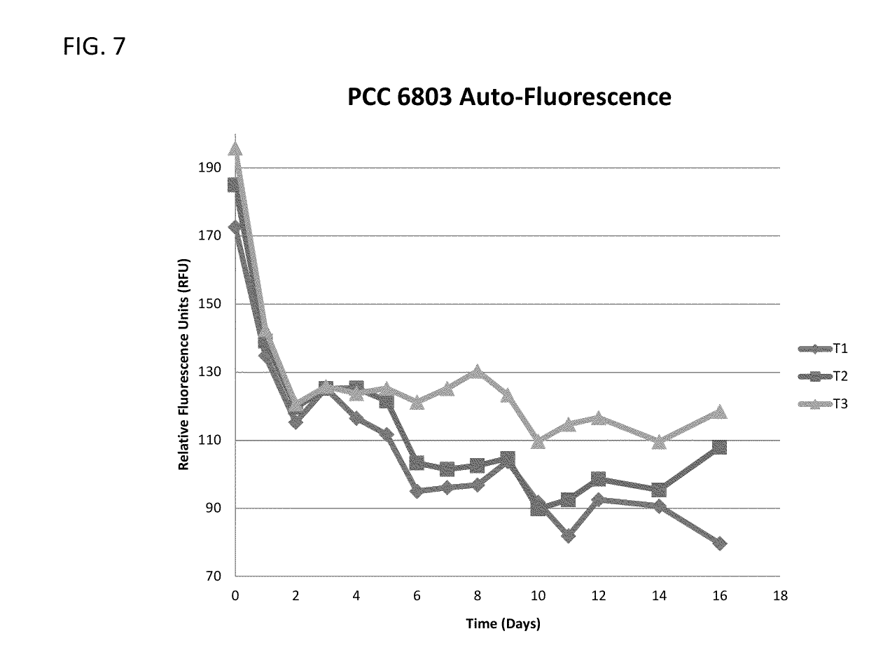

Three different cultures of PCC 6803 were started, each seeded from a 20 L starting culture and denoted T1, T2, and T3. These three cultures were monitored over a three-week time course for both OD₆₈₅ and auto-fluorescence. The three cultures, although started from the same initial batch, showed high variability in their doubling times as well as their final cell densities, with T1 having a final OD₆₈₅ of 6.287 and T3 having a final OD₆₈₅ of 10.685 (FIG. 6). In order to uncover possible distinctive markers between the cultures the flow cytometry results were analyzed for cell size and auto-fluorescence (FIG. 7). The auto-fluorescence started out at different levels for each culture, but initially dropped very quickly in the first 48 hours of the time course. This drop is most likely due to both the metabolic stress of entering logarithmic growth as well as a considerable drop in size indicated by forward scatter. The auto-fluorescence stabilized around 120 relative fluorescence units (RFUs) for all three cultures. At day six of the time course a divergence of the auto-fluorescence readings began for the three cultures, as T3 stayed steady at ˜120 RFUs while both T2 and T1 dropped over the next 10 days.

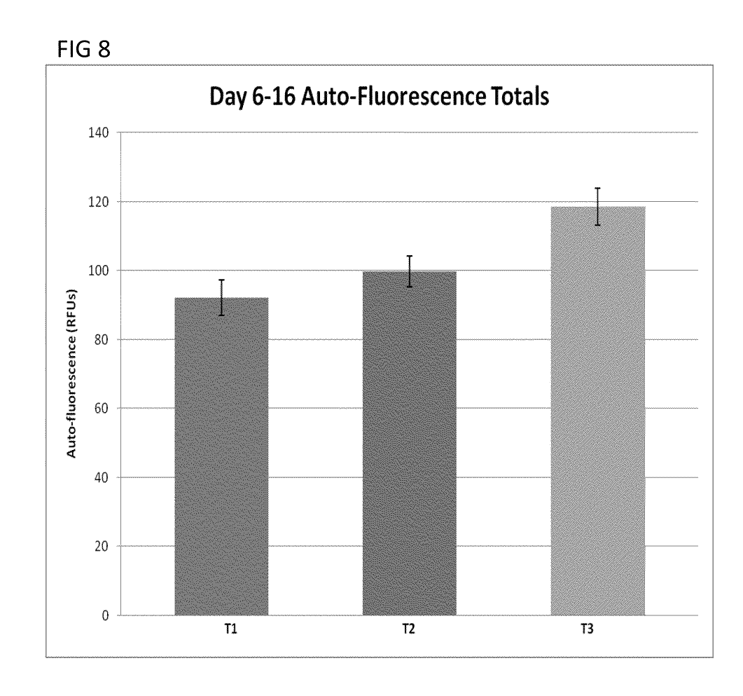

From the curves of the OD readings it appeared that the cultures were in logarithmic growth from day 6 to day 16. During this time period the average auto-fluorescence for each culture was calculated and a significant difference was seen between T3 and both T2 and T1 (FIG. 8). The auto-fluorescence detected from PCC 6803 can be used as a metric of metabolic efficiency, energy absorbed by the photosystems that exceeds the metabolic needs of the cell is released through heat and fluorescence. All three cultures were exposed to the same amount of light, so differences in auto-fluorescence may be indicative of differences in their ability to maintain efficient metabolic processes during logarithmic growth. During the logarithmic growth of the cultures the OD₆₈₅ of T3 went from about 25% more than T1 to about 50% greater than T1. During this same time period T3 had an auto-fluorescence about 25-30% higher than T1. This correlation allows for use of auto-fluorescence as a metric to sort more metabolically efficient cells from a varied starting culture and thus to increase the rate of growth.

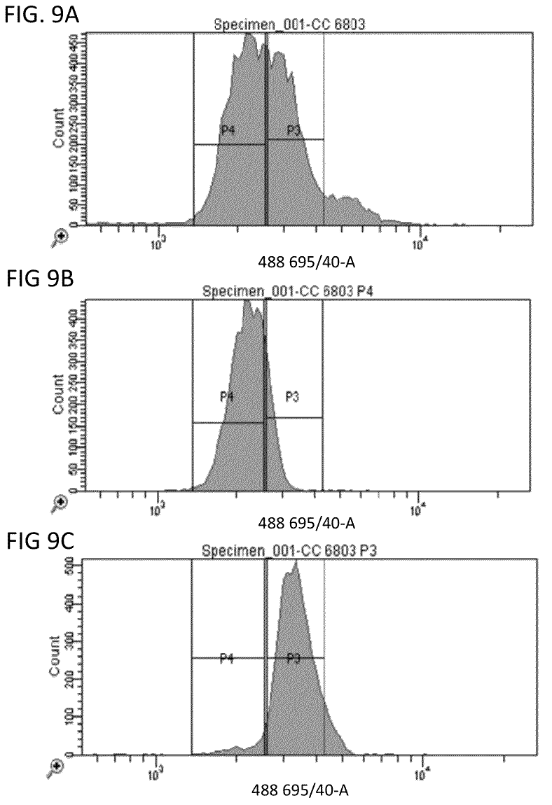

PCC 6803 Sorting: To sort PCC 6803 using flow cytometry, a gate must be set in order to determine how the cells will be separated. The flow cytometer is capable of gating both on size of cells, the side scatter vs. forward scatter plots, or by the fluorescence intensity. Tracking the mean size of the different cultures showed no significant differences, or any correlation to the growth rate of the culture. The fluorescence intensity however showed differences at both the start of growth and during the logarithmic phase of growth. At both points of variation the cultures showed differences that related to their differences in growth rate, with T3 having the highest starting fluorescence as well as the highest fluorescence during logarithmic growth. To examine these differences a Day 0 culture that was seeded from the T3 culture was sorted. The starting culture was sorted into two populations, P3 and P4. P3 was sorted to be the highest 33% of the fluorescent cells, and P4 was the lower 66% of the population (FIG. 9A-C). In this way two populations with largely different starting fluorescence levels were created and their growth was observed over 3 weeks to see if there were significant differences.

The three week time course monitoring the growth of the two sorted populations showed that initial fluorescence did not appear to dictate the rate of growth of PCC 6803. There were differences in the growth rate initially, but after 2-3 days the difference was largely eliminated. This indicates that the starting difference may have allowed the faster growth initially, but that the population was still mixed in terms of cells capable of accelerated logarithmic growth.

To determine how to isolate the accelerated population during log growth, fluorescence intensities during logarithmic growth of both T3 and T1 9the most divergent cultures) was observed, and the portion of the T3 population that appeared to be responsible for the accelerated growth rate was visualized. By overlaying the histograms of both T3 and T1 on Day 8 of the time course, where the populations differ can be determined, and markers (M2 and M3) are set to isolate these populations. When those markers were applied to the T3 cells alone they created regions 2 and 3, with region 2 being the more fluorescent population, while region 3 was the less fluorescent population. When region 2 and region 3 were isolated on the forward and side scatter plot, it was seen that they have a different size, as region 2 had a slightly larger mean forward scatter. There was however, overlap between the two populations, and sorting based solely on size would likely not provide as pure of a population of cells with accelerated growth potential.

Conclusions: These studies have identified auto-fluorescence as a metric of accelerated growth during log phase for PCC 6803. PCC 6803 mixed populations have been sorted based on this metric, resulting in isolated populations with enhanced mean auto-fluorescence. Being able to select these populations allows isolation and enrichment for faster growing cells, allowing selection of strains that can grow quickly even if they have excess metabolic stress placed upon them by engineered pathways.

### Example 4

### Expression of asbF in Synechocystis

This example describes a representative method for expression of asbF and production of 3,4-DHB in the cyanobacterium Synechocystis PCC6803. Synechocystis pCC6803 are transformed with a vector encoding asbF under standard transformation conditions. The starting vector for homologous double recombination is pMota, which was originally derived from plasmid PBSAIIKS (Vermaas, J. Appl. Phycol. 8:263-273, 1996). The vector includes a light-responsive promoter (psbAII; U.S. Pat. Publication No. 2009/0155871) and about 500 bp regions homologous to the psbAII gene upstream and downstream of the asbF sequence (e.g., SEQ ID NO: 3). Insertion of the asbF gene into all copies of the PCC6803 genome will be determined using standard PCR techniques. Complete integration is present when the psbAII gene is disrupted in all genomic copies.

In some embodiments, the cells are grown under photoautotrophic conditions, such as under exposure to 20-30 μmoles of photons/m²/min on a 16/8 hour light/dark cycle. Cells are grown in standard BG-11 media. In other embodiments, the cells are grown under photomixotrophic conditions, which include light exposure as above plus 5 mM glucose in the media. In further embodiments, the cells are grown under photoheterotrophic conditions, which are as for photomixotrophic growth as above plus 20 mM atrazine. Suitable conditions for expression of asbF and production of 3,4-DHB can be determined by one of skill in the art based on the teachings provided herein.

After a period of time (for example, 24, 36, 48, 72, 96 hours, or more) in standard PCC6803 growth media (BG-11), the cells are collected, lysed and protein content is examined by SDS-PAGE and mass spectrometry to ensure that AsbF is expressed in the soluble fraction. In some embodiments, cells are collected daily for evaluation of expression of AsbF. Fractions are collected from the culture during various phases of the growth curve (e.g., lag phase, early log phase, mid log phase, late log phase, and stationary phase) and 3,4-DHB production is analyzed by TLC, UV-Vis spectroscopy, and/or NMR, for example, as described in Example 1. In some examples, cells are collected daily for evaluation of 3,4-DHB production. Expression of detectable amounts of AsbF protein (for example, as analyzed by SDS-PAGE) and/or production of detectable amounts of 3,4-DHB (for example, as analyzed by TLC or UV-Vis spectroscopy) is considered successful expression of asbF in Synechocystis.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. A method for producing a compound utilizing dehydroshikimate as a precursor, wherein the compound is selected from 3,4-dihydroxybenzoate (3,4-DHB), catechol, cis,cis-muconate, and β-carboxy-cis,cis-muconic acid, comprising:
expressing a heterologous asbF gene in a prokaryotic cell under conditions sufficient to produce the compound; and
purifying the compound.

2. The method of claim 1, wherein the prokaryotic cell is a heterotroph.

3. The method of claim 2, wherein the heterotroph is a bacterium.

4. The method of claim 3, wherein the bacterium is E. coli, Bacillus sp., or Streptomyces sp.

5. The method of claim 1, wherein the prokaryotic cell is a phototroph.

6. The method of claim 5, wherein the phototroph is a cyanobacterium.

7. The method of claim 6, wherein the cyanobacterium is Synechocystis PCC6803, Synechocystis PCC9714, Synechococcus sp., Spirulina sp., or Anabaena variabilis.

8. The method of claim 1, wherein the asbF gene comprises an asbF gene from Bacillus thuringiensis, Bacillus cereus, or Bacillus anthracis.

9. The method of claim 1, wherein the asbF gene comprises the nucleic acid sequence set forth as SEQ ID NO: 1 or SEQ ID NO: 3.

10. The method of claim 1, wherein the asbF gene is operably linked to a promoter.

11. The method of claim 1, wherein the compound is 3,4-DHB.

12. The method of claim 1, further comprising expressing a heterologous 3,4-DHB decarboxylase gene in the prokaryotic cell, wherein the compound produced is catechol.

13. The method of claim 12, wherein the 3,4-DHB decarboxylase gene is from Klebsiella pneumoniae, Enterobacter cloacae, Lactobacillus plantarum, or Clostridium butyricum.

14. The method of claim 13, wherein the 3,4-DHB decarboxylase gene comprises the nucleic acid sequence set forth as any one of SEQ ID NOs: 4, 6, 8, and 10.

15. The method of claim 1, further comprising expressing a heterologous 3,4-DHB decarboxylase gene and a heterologous catechol 1,2-dioxygenase gene in the prokaryotic cell, wherein the compound produced is cis,cis-muconate.

16. The method of claim 15, wherein the catechol 1,2-dioxygenase gene is from Herbaspirillum seropedicae, Acinetobacter calcoaceticus, Acinetobacter radioresistens, Acinetobacter sp. ADP1, Streptomyces sp. 2065, or Pseudomonas sp.

17. The method of claim 16, wherein the catechol 1,2-dioxygenase comprises the nucleic acid sequence set forth as any one of SEQ ID NOs: 12, 14, or a combination thereof.

18. The method of 15, wherein the 3,4-DHB decarboxylase gene is from Klebsiella pneumoniae, Enterobacter cloacae, Lactobacillus plantarum, or Clostridium butyricum.

19. The method of claim 18, wherein the 3,4-DHB decarboxylase gene comprises the nucleic acid sequence set forth as any one of SEQ ID NOs: 4, 6, 8, and 10.

20. The method of claim 15, further comprising converting the cis,cis-muconate to adipic acid.

21. The method of claim 1, further comprising expressing a heterologous 3,4-DHB dioxygenase gene in the prokaryotic cell, and the compound produced is β-carboxy-cis,cis-muconic acid.

22. The method of claim 21, wherein the 3,4-DHB dioxygenase is from Streptomyces sp. 2065, Agrobacterium tumifaciens A348, Pseudomonas putida, or Herbaspirillum seropedicae.

23. The method of claim 22, wherein the 3,4-DHB dioxygenase gene comprises the nucleic acid sequence set forth as SEQ ID NOs: 16, 18, or a combination thereof.

24. The method of claim 21, further comprising converting the β-carboxy-cis,cis-muconic acid to β-carboxyadipic acid.


SEQUENCE LISTING (20 sequences)

SEQ ID NO: 1 - DNA, 843 bp, Bacillus thuringiensis
  CDS (1)..(843)
atgaaatatagcctgtgcaccattagctttcgtcatcagctgattagctttaccgatattgtgcagttcgcgtatgaaaacggctttgaaggcattgaactgtggggcacccatgcgcagaacctgtatatgcaggaatatgaaaccaccgaacgtgaactgaactgcctgaaagataaaaccctggaaatcaccatgattagcgattatctggatattagcctgagcgcggattttgaaaaaaccatcgaaaaatgcgaacagctggccattctggccaactggttcaaaaccaacaaaattcgtacctttgcgggccagaaaggcagcgcggatttcagccagcaggaacgtcaggaatacgttaaccgcattcgcatgatttgcgaactgtttgcgcagcataacatgtatgtgctgctggaaacccatccgaacaccctgaccgataccctgccgagcaccctggaactgctgggcgaagtggatcatccgaacctgaaaatcaacctggattttctgcatatttgggaaagcggtgccgatccggtggatagctttcagcagctgcgtccgtggattcagcattaccacttcaaaaacattagcagcgccgattatctgcatgtgtttgaaccgaacaacgtgtatgcggcagcgggtaaccgtaccggtatggtgccgctgttcgaaggtattgtgaactacgatgaaatcattcaggaagtgcgcgataccgatcattttgcgagcctggaatggtttggccataacgcgaaagatattctgaaagcggaaatgaaagtgctgaccaaccgtaacctggaagtggtgaccagctag

SEQ ID NO: 2 - protein, 280 aa, Bacillus thuringiensis
MKYSLCTISFRHQLISFTDIVQFAYENGFEGIELWGTHAQNLYMQEYETTERELNCLKDKTLEITMISDYLDISLSADFEKTIEKCEQLAILANWFKTNKIRTFAGQKGSADFSQQERQEYVNRIRMICELFAQHNMYVLLETHPNTLTDTLPSTLELLGEVDHPNLKINLDFLHIWESGADPVDSFQQLRPWIQHYHFKNISSADYLHVFEPNNVYAAAGNRTGMVPLFEGIVNYDEIIQEVRDTDHFASLEWFGHNAKDILKAEMKVLTNRNLEVVTS

SEQ ID NO: 3 - DNA, 840 bp, Bacillus thuringiensis
aaatactccttgtgcaccatttcctttcggcatcaattgattagttttaccgatattgtgcaatttgcctatgaaaatggctttgaaggcattgaattgtggggcacccatgcccaaaatttgtatatgcaagaatatgaaaccaccgaacgggaactgaattgcttgaaagataaaaccttggaaattaccatgatttccgattacctggacatttccttgagtgccgattttgaaaaaaccattgaaaaatgtgaacaactggccattctggccaattggtttaaaaccaacaaaattcggacctttgccggtcaaaaaggctctgccgatttttcccaacaagaacggcaagaatacgtgaatcggattcggatgatttgtgaattgtttgcccagcataacatgtatgtgttgttggaaacccatcccaataccttgaccgataccttgccctccaccttggaattgttgggcgaagtggatcatcccaatctgaaaattaacctggattttttgcatatttgggaatccggtgccgatcccgtggattcctttcaacaattgcgtccctggattcaacattatcattttaaaaatatttccagtgccgattatttgcatgtgtttgaacccaataacgtgtatgccgctgccggtaatcggaccggcatggtgcccttgtttgaaggtattgtgaactatgatgaaattattcaagaagtgcgggacaccgatcattttgccagtttggaatggtttggccataacgccaaagatattttgaaagccgaaatgaaagtgctgaccaatcggaatttggaagtggtgacctcctaa

SEQ ID NO: 4 - DNA, 1509 bp, Klebsiella pneumoniae
  CDS (1)..(1509)
atgaccgcaccgattcaggatctgcgcgacgccatcgcgctgctgcaacagcatgacaatcagtatctcgaaaccgatcatccggttgaccctaacgccgagctggccggtgtttatcgccatatcggcgcgggcggcaccgtgaagcgccccacccgcatcgggccggcgatgatgtttaacaatattaagggttatccacactcgcgcattctggtgggtatgcacgccagccgccagcgggccgcgctgctgctgggctgcgaagcctcgcagctggcccttgaagtgggtaaggcggtgaaaaaaccggtcgcgccggtggtcgtcccggccagcagcgccccctgccaggaacagatctttctggccgacgatccggattttgatttgcgcaccctgcttccggcgcacaccaacacccctatcgacgccggccccttcttctgcctgggcctggcgctggccagcgatcccgtcgacgcctcgctgaccgacgtcaccatccaccgcttgtgcgtccagggccgggatgagctgtcgatgtttcttgccgccggccgccatatcgaagtgtttcgccaaaaggccgaggccgccggcaaaccgctgccgataaccatcaatatgggtctcgatccggccatctatattggcgcctgcttcgaagcccctaccacgccgttcggctataatgagctgggcgtcgccggcgcgctgcgtcaacgtccggtggagctggttcagggcgtcagcgtcccggagaaagccatcgcccgcgccgagatcgttatcgaaggtgagctgttgcctggcgtgcgcgtcagagaggatcagcacaccaatagcggccacgcgatgccggaatttcctggctactgcggcggcgctaatccgtcgctgccggtaatcaaagtcaaagcagtgaccatgcgaaacaatgcgattctgcagaccctggtgggaccgggggaagagcataccaccctcgccggcctgccaacggaagccagtatctggaatgccgtcgaggccgccattccgggctttttacaaaatgtctacgcccacaccgcgggtggcggtaagttcctcgggatcctgcaggtgaaaaaacgtcaacccgccgatgaaggccggcaggggcaggccgcgctgctggcgctggcgacctattccgagctaaaaaatattattctggttgatgaagatgtcgacatctttgacagcgacgatatcctgtgggcgatgaccacccgcatgcagggggacgtcagcattacgacaatccccggcattcgcggtcaccagctggatccgtcccagacgccggaatacagcccgtcgatccgtggaaatggcatcagctgcaagaccatttttgactgcacggtcccctgggcgctgaaatcgcactttgagcgcgcgccgtttgccgacgtcgatccgcgtccgtttgcaccggagtatttcgcccggctggaaaaaaaccagggtagcgcaaaataa

SEQ ID NO: 5 - protein, 502 aa, Klebsiella pneumoniae
MTAPIQDLRDAIALLQQHDNQYLETDHPVDPNAELAGVYRHIGAGGTVKRPTRIGPAMMFNNIKGYPHSRILVGMHASRQRAALLLGCEASQLALEVGKAVKKPVAPVVVPASSAPCQEQIFLADDPDFDLRTLLPAHTNTPIDAGPFFCLGLALASDPVDASLTDVTIHRLCVQGRDELSMFLAAGRHIEVFRQKAEAAGKPLPITINMGLDPAIYIGACFEAPTTPFGYNELGVAGALRQRPVELVQGVSVPEKAIARAEIVIEGELLPGVRVREDQHTNSGHAMPEFPGYCGGANPSLPVIKVKAVTMRNNAILQTLVGPGEEHTTLAGLPTEASIWNAVEAAIPGFLQNVYAHTAGGGKFLGILQVKKRQPADEGRQGQAALLALATYSELKNIILVDEDVDIFDSDDILWAMTTRMQGDVSITTIPGIRGHQLDPSQTPEYSPSIRGNGISCKTIFDCTVPWALKSHFERAPFADVDPRPFAPEYFARLEKNQGSAK

SEQ ID NO: 6 - DNA, 1667 bp, Enterobacter cloacae
  CDS (135)..(1622)
acgcatcagacgaaattgcatgacgaagtcccgcgaatttgataataaaattctatcaaaatagcatcaatgatgcaattgatgctatctgtcgttcgcccaacaatggaggtcagccattaagggagaaaaacatgcaaaaccccatcaacgatctcagaagcgccatcgcgttgctgcaacgccatccaggtcactatatcgaaaccgatcacccggtagatcccaatgctgaactggcgggcgtctaccgccatatcggcgcgggcggtaccgtaaaacgccccacccgcacgggcccggccatgatgttcaatagcgtgaagggctaccctggctcccgcatcctggtaggtatgcacgccagccgggaaagagcggcgcttctgctgggctgtgtaccctcgaagctggcacagcacgttggtcaggcggtgaaaaacccggttgcaccggtggtggttccggcctcgcaggcaccgtgccaggagcaggtcttttacgccgacgatccggactttgacctgcgtaagctgcttccggccccgaccaacacgccgattgatgcaggcccgttcttctgtctggggctggtactggcaagcgatccggaagatacctcgctgaccgatgtgaccattcaccgtctctgtgtgcaggagcgagatgaactctctatgttccttgccgccggccgccatatcgaagtctttcgcaagaaggccgaagcggcgggcaaaccgctgccggtaaccatcaatatgggacttgacccggctatctacattggggcctgtttcgaagcgccaaccacgccattcggttacaacgagcttggcgttgccggggcattacgccagcaaccggtggagctggtacagggcgtggcggtaaaagagaaagcgatcgcgcgggcggaaatcatcatcgagggcgaactgcttcccggcgtgcgcgtaagagaagatcagcacaccaacaccggccacgccatgccggagttcccgggctactgcggcgaggcgaatccgtctctgccggtgatcaaagtgaaagccgtgacgatgcgaaaccacgcgatcctgcagacgctggtgggcccgggcgaagagcacaccacgcttgccggtttgccgaccgaggccagcattcgcaacgcggtcgaagaggccattcccggctttctgcaaaacgtttacgcccacaccgccggaggcggtaaattcctcggcattttacaggtgaaaaaacgccagccgtcagacgaaggacgtcagggccaggcggcacttatcgccctggccacctattccgagctgaaaaacattatcctcgtggatgaagacgtggatatcttcgacagcgacgatatcctgtgggcaatgaccacccgcatgcagggcgatgtgagcatcaccacgcttccggggatccgcggccaccagctggatccgtcgcagtcaccggactacagcacctcgatccgtggaaacggcatttcctgcaagactatcttcgactgcacggtgccgtgggcgctgaaggcgcggtttgaacgggcgccgttcatggaggttgaccccacaccgtgggcgccggagctgttcagcgataaaaaatagaccgtcgtcgccgtttcttcgccccaccgggtgaagaaacgcaag

SEQ ID NO: 7 - protein, 495 aa, Enterobacter cloacae
MQNPINDLRSAIALLQRHPGHYIETDHPVDPNAELAGVYRHIGAGGTVKRPTRTGPAMMFNSVKGYPGSRILVGMHASRERAALLLGCVPSKLAQHVGQAVKNPVAPVVVPASQAPCQEQVFYADDPDFDLRKLLPAPTNTPIDAGPFFCLGLVLASDPEDTSLTDVTIHRLCVQERDELSMFLAAGRHIEVFRKKAEAAGKPLPVTINMGLDPAIYIGACFEAPTTPFGYNELGVAGALRQQPVELVQGVAVKEKAIARAEIIIEGELLPGVRVREDQHTNTGHAMPEFPGYCGEANPSLPVIKVKAVTMRNHAILQTLVGPGEEHTTLAGLPTEASIRNAVEEAIPGFLQNVYAHTAGGGKFLGILQVKKRQPSDEGRQGQAALIALATYSELKNIILVDEDVDIFDSDDILWAMTTRMQGDVSITTLPGIRGHQLDPSQSPDYSTSIRGNGISCKTIFDCTVPWALKARFERAPFMEVDPTPWAPELFSDKK

SEQ ID NO: 8 - DNA, 1482 bp, Lactobacillus plantarum
  CDS (1)..(1482)
atgaatgaaatggcagaacaaccatgggatttgcgtcgcgtgcttgatgagatcaaggatgatccaaagaactatcatgaaactgacgtcgaagttgatccaaatgcggaactttctggtgtttatcggtatatcggtgctggtgggaccgttcaacggccaacgcaagagggtccagcaatgatgtttaacaacgttaaggggtttcctgatacgcgggtcttgactggattgatggcgagtcgccggcgcgttggtaagatgttccaccacgattatcagacgttagggcaatacttgaacgaagcagtctctaatccagtggcgccagaaacggttgctgaagcggatgcgccagctcacgatgtcgtttataaagcgacggatgaaggctttgatattcgtaagttagtggcagcaccaacgaatacgccccaagatgctggaccatatattacggtcggtgtggtgtttggctcaagcatggacaagtctaagagtgatgtgacgattcaccgaatggtccttgaagataaggataagttagggatttatatcatgcctggcggtcggcacattggtgcgtttgcggaagagtatgagaaagctaacaagccaatgccaattacaattaatattggtttggatccagccattacgattggtgcaactttcgaaccaccgaccacgccattcggttataacgaattaggtgttgctggtgcgattcggaaccaagctgttcaattagttgacggggtgaccgtcgatgaaaaggcgattgcgcgttctgaatatacgcttgaggggtacattatgcctaacgaacgtattcaggaagatatcaatacgcatacgggcaaggcgatgcctgaattcccgggttatgatggtgacgccaacccagctttacaagtgattaaggtgacggcggtgactcatcggaagaatgccatcatgcaaagcgtgattggaccatccgaagaacatgtcagcatggcgggaattccaactgaagctagtatcttacaattggttaaccgtgccattcctggtaaagtgacgaatgtttataatccgccggctggtggtggtaagttgatgaccatcatgcagattcacaaggataatgaagcggatgaaggaattcaacggcaagctgccttgcttgcgttctcagcctttaaggaattgaagactgttatcctggttgatgaagatgttgatatttttgatatgaatgatgtgatttggacgatgaatacccgtttccaagccgatcaggacttgatggtcttatcaggcatgcggaatcatccgttggacccatcggaacgcccacaatatgatccaaagtcgattcgtttccgtgggatgagttctaaactagtgattgatggcaccgtaccattcgatatgaaggaccaatttgaacgggcccaattcatgaaagtggctgactgggagaagtatttgaagtaa

SEQ ID NO: 9 - protein, 493 aa, Lactobacillus plantarum
MNEMAEQPWDLRRVLDEIKDDPKNYHETDVEVDPNAELSGVYRYIGAGGTVQRPTQEGPAMMFNNVKGFPDTRVLTGLMASRRRVGKMFHHDYQTLGQYLNEAVSNPVAPETVAEADAPAHDVVYKATDEGFDIRKLVAAPTNTPQDAGPYITVGVVFGSSMDKSKSDVTIHRMVLEDKDKLGIYIMPGGRHIGAFAEEYEKANKPMPITINIGLDPAITIGATFEPPTTPFGYNELGVAGAIRNQAVQLVDGVTVDEKAIARSEYTLEGYIMPNERIQEDINTHTGKAMPEFPGYDGDANPALQVIKVTAVTHRKNAIMQSVIGPSEEHVSMAGIPTEASILQLVNRAIPGKVTNVYNPPAGGGKLMTIMQIHKDNEADEGIQRQAALLAFSAFKELKTVILVDEDVDIFDMNDVIWTMNTRFQADQDLMVLSGMRNHPLDPSERPQYDPKSIRFRGMSSKLVIDGTVPFDMKDQFERAQFMKVADWEKYLK

SEQ ID NO: 10 - DNA, 1464 bp, Clostridium butyricum
  CDS (1)..(1464)
atgagcaataaagtatatgatcttagaagtgcattagaattattaaaaactctgccaggacaattgatagaaacagatgtggaagtagattcaatggcggaattagcaggagtttatcgttatgttggtgctggtggaacggttcagcgtcctacaaaagaaggaccagcaatgatttttaataatataaaaggacacaaagatgcaagagtattaattggattacttgcaagccgtagacgagtggcagcacttttagattgtgaacctgaaaatttaggaaagttattatatagaagtgtcgataatccaattgccccagtacttacaaacgcaaaattacctttatgtcagcaggtcgttcataaagcaacagatccagattttgatttaaataaattagtaccggcaccaacaaatacacctgatgatgctgggccttatattacacttggaatgtgttatgcaagtcatccagatacaaaatttagtgatgttacgattcatcgtttatgcattcaggggaaggatgaactttcaatattctttactccaggagcaaggcacataggtgctatggcagaaagagcagaagaattaggacaaaatcttcctatttcaataagtataggtgtagatcctgctatagaaataggttcatgttttgaaccaccaactactccattaggatatgatgagttatcagttgcaggagcactaagaggaaagccagtggagctttgcaattgtattacagtaaatgaaagagctattgcaaatgccgaatatgttattgaaggtgaagttatacctaatttaagagtacaggaagataaaaacagcaatacaggatatgctatgccggaatttcctgggtatacaggaccagcaagcgatcaatgttggatgataaaggttaaagctgttacacatagagaaaatccaattatgcaaacatgtataggtccaagtgaagagcacgtatcaatggcaggtataccaacagaagctagtatttatggaatgattgaaaaagcaatgccaggaagattacaaaatgtatactgctgttcatctggtggtggaaaattcatggctgtattacagtttaaaaagactgttgcaagtgatgaagggcgtcaaagacaggctgcattattagcattttcagcattcagtgaacttaaaaatatattcattgtagatgaagatgtggactgttttgatatgaatgatgttttatgggcaatgaatacacgatttcagggagatgcagatattataacaattcctggagtgagatgtcatccacttgatccatcaaatgatccagattattctccaaccataaaaaatcatggaattgcatgtaaaacaatatttgattgtactgtaccttttcatatgaaagaaagatttaaaagagctaaatttatggaagttgatccagagcattggttataa

SEQ ID NO: 11 - protein, 487 aa, Clostridium butyricum
MSNKVYDLRSALELLKTLPGQLIETDVEVDSMAELAGVYRYVGAGGTVQRPTKEGPAMIFNNIKGHKDARVLIGLLASRRRVAALLDCEPENLGKLLYRSVDNPIAPVLTNAKLPLCQQVVHKATDPDFDLNKLVPAPTNTPDDAGPYITLGMCYASHPDTKFSDVTIHRLCIQGKDELSIFFTPGARHIGAMAERAEELGQNLPISISIGVDPAIEIGSCFEPPTTPLGYDELSVAGALRGKPVELCNCITVNERAIANAEYVIEGEVIPNLRVQEDKNSNTGYAMPEFPGYTGPASDQCWMIKVKAVTHRENPIMQTCIGPSEEHVSMAGIPTEASIYGMIEKAMPGRLQNVYCCSSGGGKFMAVLQFKKTVASDEGRQRQAALLAFSAFSELKNIFIVDEDVDCFDMNDVLWAMNTRFQGDADIITIPGVRCHPLDPSNDPDYSPTIKNHGIACKTIFDCTVPFHMKERFKRAKFMEVDPEHWL

SEQ ID NO: 12 - DNA, 945 bp, Acinetobacter radioresistens
  CDS (1)..(945)
atgaccgcagccaatgtgaaaattctgaataccgaagaagtgcagaattttattaatctgctgagtggtctggaacaagaaggtggtaatccgcgtattaaacaaattattcatcgtgttgtgagcgacctgtttaaaagcattgaggatctggaaattaccagtgatgaatattgggcagccattgcatatctgaatcagctgggcaccagccatgaagcaggtctgctgagtccgggtctgggttttgatcattttctggatatgcgtatggatgccattgatgcagcactgggtattgataatccgacaccgcgtaccattgaaggtccgctgtatgttgcaggcgcaccggttagccagggttttgcacgtatggatgatggtagcgatccgaatggtcataccctgattctgcatggcaccatttataatgcagatggtcagccgattccgaatgcacaggttgaaatttggcatgcaaataccaaaggcttttatagccattttgatccgaccggtgaacagaccccgtttaatatgcgtcgtaccattatgaccgatgcacagggtcattatcgtgttcagaccattctgccgagcggttatggttgtccgccgaatggtccgacccagcaactgctgaatcagctgggtcgtcatggtaatcgtccggcacatattcatttttttgttagcgcagatggctatcgtaaactgaccacccagattaatgttgcgggtgatccgtatacctatgatgattttgcatttgcaacccgtgaaggtctggttgttgaagccattgaacataccgatccggcaaccagccagcgtaatggtgttgaaggtccgtttgcagaaatggtttttgatctgaaactgagccgtctggttgatggtgttgataatcaggttgttgatcgtccgcgtctgcaggcataa

SEQ ID NO: 13 - protein, 314 aa, Acinetobacter radioresistens
MTAANVKILNTEEVQNFINLLSGLEQEGGNPRIKQIIHRVVSDLFKSIEDLEITSDEYWAAIAYLNQLGTSHEAGLLSPGLGFDHFLDMRMDAIDAALGIDNPTPRTIEGPLYVAGAPVSQGFARMDDGSDPNGHTLILHGTIYNADGQPIPNAQVEIWHANTKGFYSHFDPTGEQTPFNMRRTIMTDAQGHYRVQTILPSGYGCPPNGPTQQLLNQLGRHGNRPAHIHFFVSADGYRKLTTQINVAGDPYTYDDFAFATREGLVVEAIEHTDPATSQRNGVEGPFAEMVFDLKLSRLVDGVDNQVVDRPRLQA

SEQ ID NO: 14 - DNA, 918 bp, Acinetobacter radioresistens
  CDS (1)..(918)
aatcgccagcagattgatgcactggttaaacaaatgaatgtggataccgcaaaaggtccggttgatgaacgtattcagcaggttgttgttcgtctgctgggtgacctgtttcaggccattgaggatctggatattcagccgagcgaagtttggaaaggtctggaatatctgaccgatgcaggtcaggcaaatgaactgggtctgctggcagcaggtctgggtctggaacattatctggatctgcgtgcagatgaagcagatgcaaaagcaggtattaccggtggtacaccgcgtaccattgaaggtccgctgtatgttgcaggcgcaccggaaagcgttggttttgcacgtatggatgatggtagcgaaagcgataaagttgataccctgattattgaaggcaccgttaccgataccgaaggcaacattattgaaggtgccaaagttgaagtgtggcatgcaaatagcctgggtaattatagcttttttgataaaagccagagcgattttaatctgcgtcgtaccattctgaccgatgtgaatggtaaatatgtggcactgaccaccatgccggttggttatggttgtccgccggaaggcaccacccaggcactgctgaataaactgggtcgtcatggtaatcgtccgagccatgttcattattttgttagcgcaccgggttatcgtaaactgaccacccagtttaatattgaaggtgatgaatatctgtgggatgattttgcatttgcaacccgtgatggtctggttgcaaccgcaaccgatgttaccgatgaagcagaaattgcccgtcgtgaactggataaaccgtttaaacacattacctttaatgtggaactggtgaaagaagcagaagcagcaccgagcagcgaagttgaacgtcgtcgtgcaagcgcataa

SEQ ID NO: 15 - protein, 305 aa, Acinetobacter radioresistens
NRQQIDALVKQMNVDTAKGPVDERIQQVVVRLLGDLFQAIEDLDIQPSEVWKGLEYLTDAGQANELGLLAAGLGLEHYLDLRADEADAKAGITGGTPRTIEGPLYVAGAPESVGFARMDDGSESDKVDTLIIEGTVTDTEGNIIEGAKVEVWHANSLGNYSFFDKSQSDFNLRRTILTDVNGKYVALTTMPVGYGCPPEGTTQALLNKLGRHGNRPSHVHYFVSAPGYRKLTTQFNIEGDEYLWDDFAFATRDGLVATATDVTDEAEIARRELDKPFKHITFNVELVKEAEAAPSSEVERRRASA

SEQ ID NO: 16 - DNA, 606 bp, Streptomyces sp. 2065
  CDS (1)..(606)
atgacgaccatcgacacgagccgcccggagtccgtgcagccgaccccgtcgcacacggtcggccccttctacggctacgcgctgcccttccccggcggcggcgacatcgccccggtcggccaccccgacacgatcaccgtccagggctacatctacgacggcgaaggcaaaccactccccgacgccttcgtggaactctggggccccgaccccgagggcaacctctccacgaccgacggctcgatccggcgcgacccggccagcggcggctatctcggccgcaacggcgtggagttcaccggctggggccgcatccagacggacgccaacggccactggtacgcacggacgctgcgcccgggagcgcgcggccaaagcgccccgtacctgagcgcgtgcgtcttcgcgcgcggactgctggtgcacctcttcacccgcatctacctcccgggcgacgagcccacgctcaccgcggacccgctgctgtccgggctcgacccggcgcggcgcggcacgctgatcgcgcgggacgagggcaggggcacataccgtttcgacatccgccttcagggcgaaggcgagacggtattcctggagttccagtga

SEQ ID NO: 17 - protein, 201 aa, Streptomyces sp. 2065
MTTIDTSRPESVQPTPSHTVGPFYGYALPFPGGGDIAPVGHPDTITVQGYIYDGEGKPLPDAFVELWGPDPEGNLSTTDGSIRRDPASGGYLGRNGVEFTGWGRIQTDANGHWYARTLRPGARGQSAPYLSACVFARGLLVHLFTRIYLPGDEPTLTADPLLSGLDPARRGTLIARDEGRGTYRFDIRLQGEGETVFLEFQ

SEQ ID NO: 18 - DNA, 774 bp, Streptomyces sp. 2065
  CDS (1)..(774)
atgactctcacccagcacgacatcgacctcgaaatagcggccgagcacgcgacgtacgagaagcgggtcgccgacggcgcgccggtcgagcaccacccgcgccgcgactacgccccgtaccgctcctccacgctccgccacccgaaacagccgccggtcaccatcgacgtctccaaggaccccgaactggtggagctggcctcgcccgcgttcggcgagcgggacatcacggagatcgacaacgacctgacccggcagcacaacggcgagccgatcggggagcggatcaccgtctccggacggctgttggaccgtgacgggcgcccgatccgcggccagctggtcgagatctggcaggcgaactcggccggccgctacgcccaccagcgcgagcagcacgacgccccgctggaccccaacttcactggtgtgggccgcacgttgaccgacgacgagggcgggtaccacttcacgaccgtccagccgggcccctacccctggcgcaaccacgtcaacgcctggcgcccggcgcacatccacttctcgatgttcggctcggcgttcacgcaacggctcgtcacgcagatgtacttcccgagcgacccgctgttcccgtacgacccgatcatccagtcggtgacggacgacgcggcccgccaacggctcgtcgcgacgtacgaccacagcctgtcggtgcccgagttctcgatgggctaccactgggacatcgtgctcgacggcccgcacgccacctggatcgaagaaggacgctga

SEQ ID NO: 19 - protein, 257 aa, Streptomyces sp. 2065
MTLTQHDIDLEIAAEHATYEKRVADGAPVEHHPRRDYAPYRSSTLRHPKQPPVTIDVSKDPELVELASPAFGERDITEIDNDLTRQHNGEPIGERITVSGRLLDRDGRPIRGQLVEIWQANSAGRYAHQREQHDAPLDPNFTGVGRTLTDDEGGYHFTTVQPGPYPWRNHVNAWRPAHIHFSMFGSAFTQRLVTQMYFPSDPLFPYDPIIQSVTDDAARQRLVATYDHSLSVPEFSMGYHWDIVLDGPHATWIEEGR

SEQ ID NO: 20 - DNA, 17290 bp, Artificial Sequence
  Vector for cyanobacterial expression including AsbF, 3,4-DHB decarboxlase, and catechol 1,2-dioxygenase coding sequences
tcttccgcttcctcgctcactgactcgctgcgctcggtcgttcggctgcggcgagcggtatcagctcactcaaaggcggtaatacggttatccacagaatagaaggcgaaggagcgagtgactgagcgacgcgagccagcaagccgacgccgctcgccatagtcgagtgagtttccgccattatgccaataggtgtcttacaggggataacgcaggaaagaacatgtgagcaaaaggccagcaaaaggccaggaaccgtaaaaaggccgcgttgctggcgtttttccataggctccgcccgtcccctattgcgtcctttcttgtacactcgttttccggtcgttttccggtccttggcatttttccggcgcaacgaccgcaaaaaggtatccgaggcgggccctgacgagcatcacaaaaatcgacgctcaagtcagaggtggcgaaacccgacaggactataaagataccaggcgtttccccctggaagctccctcgtggggactgctcgtagtgtttttagctgcgagttcagtctccaccgctttgggctgtcctgatatttctatggtccgcaaagggggaccttcgagggagcaccgctctcctgttccgaccctgccgcttaccggatacctgtccgcctttctcccttcgggaagcgtggcgctttctcatagctcacgctgtaggtatctcagcgagaggacaaggctgggacggcgaatggcctatggacaggcggaaagagggaagcccttcgcaccgcgaaagagtatcgagtgcgacatccatagagtgttcggtgtaggtcgttcgctccaagctgggctgtgtgcacgaaccccccgttcagcccgaccgctgcgccttatccggtaactatcgtcttgagtccaacaagccacatccagcaagcgaggttcgacccgacacacgtgcttggggggcaagtcgggctggcgacgcggaataggccattgatagcagaactcaggttcccggtaagacacgacttatcgccactggcagcagccactggtaacaggattagcagagcgaggtatgtaggcggtgctacagagttcttgaagtggtgggggccattctgtgctgaatagcggtgaccgtcgtcggtgaccattgtcctaatcgtctcgctccatacatccgccacgatgtctcaagaacttcaccacccctaactacggctacactagaagaacagtatttggtatctgcgctctgctgaagccagttaccttcggaaaaagagttggtagctcttgatccggcaaacggattgatgccgatgtgatcttcttgtcataaaccatagacgcgagacgacttcggtcaatggaagcctttttctcaaccatcgagaactaggccgtttgaaaccaccgctggtagcggtggtttttttgtttgcaagcagcagattacgcgcagaaaaaaaggatctcaagaagatcctttgatcttttctacggggtctttggtggcgaccatcgccaccaaaaaaacaaacgttcgtcgtctaatgcgcgtctttttttcctagagttcttctaggaaactagaaaagatgccccagtgacgctcagtggaacgaaaactcacgttaagggattttggtcatgagattatcaaaaaggatcttcacctagatccttttaaattaaaaatgaagttttactgcgagtcaccttgcttttgagtgcaattccctaaaaccagtactctaatagtttttcctagaagtggatctaggaaaatttaatttttacttcaaaaaaatcaatctaaagtatatatgagtaaacttggtctgacagttaccaatgcttaatcagtgaggcacctatctcagcgatctgtctatttcgttcatccatttagttagatttcatatatactcatttgaaccagactgtcaatggttacgaattagtcactccgtggatagagtcgctagacagataaagcaagtaggttagttgcctgactccccgtcgtgtagataactacgatacgggagggcttaccatctggccccagtgctgcaatgataccgcgagacccacgctcaccggcatcaacggactgaggggcagcacatctattgatgctatgccctcccgaatggtagaccggggtcacgacgttactatggcgctctgggtgcgagtggccgtccagatttatcagcaataaaccagccagccggaagggccgagcgcagaagtggtcctgcaactttatccgcctccatccagtctattaattgttgccggaggtctaaatagtcgttatttggtcggtcggccttcccggctcgcgtcttcaccaggacgttgaaataggcggaggtaggtcagataattaacaacggccgaagctagagtaagtagttcgccagttaatagtttgcgcaacgttgttgccattgctacaggcatcgtggtgtcacgctcgtcgtttggtatggcttcatcttcgatctcattcatcaagcggtcaattatcaaacgcgttgcaacaacggtaacgatgtccgtagcaccacagtgcgagcagcaaaccataccgaagtatcagctccggttcccaacgatcaaggcgagttacatgatcccccatgttgtgcaaaaaagcggttagctccttcggtcctccgatcgttgtcagaagtaaagtcgaggccaagggttgctagttccgctcaatgtactagggggtacaacacgttttttcgccaatcgaggaagccaggaggctagcaacagtcttcattgttggccgcagtgttatcactcatggttatggcagcactgcataattctcttactgtcatgccatccgtaagatgcttttctgtgactggtgagtactcacaaccggcgtcacaatagtgagtaccaataccgtcgtgacgtattaagagaatgacagtacggtaggcattctacgaaaagacactgaccactcatgagtaccaagtcattctgagaatagtgtatgcggcgaccgagttgctcttgcccggcgtcaatacgggataataccgcgccacatagcagaactttaaaagtgctggttcagtaagactcttatcacatacgccgctggctcaacgagaacgggccgcagttatgccctattatggcgcggtgtatcgtcttgaaattttcacgtcatcattggaaaacgttcttcggggcgaaaactctcaaggatcttaccgctgttgagatccagttcgatgtaacccactcgtgcacccaactgatcttcagtagtaaccttttgcaagaagccccgcttttgagagttcctagaatggcgacaactctaggtcaagctacattgggtgagcacgtgggttgactagaagagcatcttttactttcaccagcgtttctgggtgagcaaaaacaggaaggcaaaatgccgcaaaaaagggaataagggcgacacggaaatgttgaatactctcgtagaaaatgaaagtggtcgcaaagacccactcgtttttgtccttccgttttacggcgttttttcccttattcccgctgtgcctttacaacttatgagatactcttcctttttcaatattattgaagcatttatcagggttattgtctcatgagcggatacatatttgaatgtatttagaaaaataaacaaataggggtatgagaaggaaaaagttataataacttcgtaaatagtcccaataacagagtactcgcctatgtataaacttacataaatctttttatttgtttatccccttccgcgcacatttccccgaaaagtgccacctgacgtctaagaaaccattattatcatgacattaacctataaaaataggcgtatcacgaggccctttcgaaggcgcgtgtaaaggggcttttcacggtggactgcagattctttggtaataatagtactgtaattggatatttttatccgcatagtgctccgggaaagctctcgcgcgtttcggtgatgacggtgaaaacctctgacacatgcagctcccggagacggtcacagcttgtctgtaagcggatgccgggagcagacaagccagagcgcgcaaagccactactgccacttttggagactgtgtacgtcgagggcctctgccagtgtcgaacagacattcgcctacggccctcgtctgttcggcgtcagggcgcgtcagcgggtgttggcgggtgtcggggctggcttaactatgcggcatcagagcagattgtactgagagtgcaccataaaattgtaaacggcagtcccgcgcagtcgcccacaaccgcccacagccccgaccgaattgatacgccgtagtctcgtctaacatgactctcacgtggtattttaacatttgcttaatattttgttaaaattcgcgttaaatttttgttaaatcagctcattttttaaccaataggccgaaatcggcaaaatcccttataaatcaaaagaataaattataaaacaattttaagcgcaatttaaaaacaatttagtcgagtaaaaaattggttatccggctttagccgttttagggaatatttagttttcttatgcccgagatagggttgagtgttgttccagtttggaacaagagtccactattaaagaacgtggactccaacgtcaaagggcgaaaaaccgtctatcagggccgggctctatcccaactcacaacaaggtcaaaccttgttctcaggtgataatttcttgcacctgaggttgcagtttcccgctttttggcagatagtcccggatggcccactacgtgaaccatcacccaaatcaagttttttggggtcgaggtgccgtaaagcactaaatcggaaccctaaagggagcccccgatttagagctaccgggtgatgcacttggtagtgggtttagttcaaaaaaccccagctccacggcatttcgtgatttagccttgggatttccctcgggggctaaatctccttgacggggaaagccggcgaacgtggcgagaaaggaagggaagaaagcgaaaggagcgggcgctagggcgctggcaagtgtagcggtcacgctgcgcgtgaactgcccctttcggccgcttgcaccgctctttccttcccttctttcgctttcctcgcccgcgatcccgcgaccgttcacatcgccagtgcgacgcgcaaaccaccacacccgccgcgcttaatgcgccgctacagggcgcgtactatggttgctttgacgtatgcggtgtgaaataccgcacagatgcgtaaggagaattggtggtgtgggcggcgcgaattacgcggcgatgtcccgcgcatgataccaacgaaactgcatacgccacactttatggcgtgtctacgcattcctcttaataccgcatcaggcgccattcgccattcaggctgcgcaactgttgggaagggcgatcggtgcgggcctcttcgctattacgccagctggcgaaagggggttatggcgtagtccgcggtaagcggtaagtccgacgcgttgacaacccttcccgctagccacgcccggagaagcgataatgcggtcgaccgctttcccccatgtgctgcaaggcgattaagttgggtaacgccagggttttcccagtcacgacgttgtaaaacgacggccagtgccaagcttaaggtgcacggcccacgttacacgacgttccgctaattcaacccattgcggtcccaaaagggtcagtgctgcaacattttgctgccggtcacggttcgaattccacgtgccgggtgcaggccactagtacttctcgagctctgtacatgtccgcggtcgcgacgtacgcgtatcgatggcgccagctgcagagcgttccagtggatatttgctgggggccggtgatcatgaagagctcgagacatgtacaggcgccagcgctgcatgcgcatagctaccgcggtcgacgtctcgcaaggtcacctataaacgacccccttaatgaaacattgtggcggaacccagggacaatgtgaccaaaaaattcagggatatcaataagtattaggtatatggatcataattgtatgcccgactaaattactttgtaacaccgccttgggtccctgttacactggttttttaagtccctatagttattcataatccatatacctagtattaacatacgggctgatttgcttaaactgactgaccactgaccttaagagtaatggcgtgcaaggcccagtgatcaatttcattatttttcattatttcatctccattgtccctgaaaacgaatttgactgactggtgactggaattctcattaccgcacgttccgggtcactagttaaagtaataaaaagtaataaagtagaggtaacagggacttaatcagttgtgtcgcccctctacacagcccagaactatggtaaaggcgcacgaaaaaccgccaggtaaactcttctcaacccccaaaacgccctctgtttttagtcaacacagcggggagatgtgtcgggtcttgataccatttccgcgtgctttttggcggtccatttgagaagagttgggggttttgcgggagacaaaacccatggaaaaaacgacaattacaagaaagtaaaacttatgtcatctataagcttcgtgtatattaacttcctgttacaaagctttacaaaactctcattgggtaccttttttgctgttaatgttctttcattttgaatacagtagatattcgaagcacatataattgaaggacaatgtttcgaaatgttttgagagtataatcctttagactaagtttagtcagttccaatctgaacatcgacaaatacataaggaattataaccatatgcatcatcatcatcaccatgattacgataattaggaaatctgattcaaatcagtcaaggttagacttgtagctgtttatgtattccttaatattggtatacgtagtagtagtagtggtactaatgctatttcccaccaccgaaaatttgtattttcagggctccaaatactccttgtgcaccatttcctttcggcatcaattgattagttttaccgatattgtgcaattaagggtggtggcttttaaacataaaagtcccgaggtttatgaggaacacgtggtaaaggaaagccgtagttaactaatcaaaatggctataacacgttaatgcctatgaaaatggctttgaaggcattgaattgtggggcacccatgcccaaaatttgtatatgcaagaatatgaaaccaccgaacgggaactgaattgcacggatacttttaccgaaacttccgtaacttaacaccccgtgggtacgggttttaaacatatacgttcttatactttggtggcttgcccttgacttaacgttgaaagataaaaccttggaaattaccatgatttccgattacctggacatttccttgagtgccgattttgaaaaaaccattgaaaaatgtgaacaactggaactttctattttggaacctttaatggtactaaaggctaatggacctgtaaaggaactcacggctaaaacttttttggtaactttttacacttgttgaccccattctggccaattggtttaaaaccaacaaaattcggacctttgccggtcaaaaaggctctgccgatttttcccaacaagaacggcaagaatacgtgaaggtaagaccggttaaccaaattttggttgttttaagcctggaaacggccagtttttccgagacggctaaaaagggttgttcttgccgttcttatgcactttcggattcggatgatttgtgaattgtttgcccagcataacatgtatgtgttgttggaaacccatcccaataccttgaccgataccttgccctccaccttgagcctaagcctactaaacacttaacaaacgggtcgtattgtacatacacaacaacctttgggtagggttatggaactggctatggaacgggaggtggaacgaattgttgggcgaagtggatcatcccaatctgaaaattaacctggattttttgcatatttgggaatccggtgccgatcccgtggattcctttcaacaatcttaacaacccgcttcacctagtagggttagacttttaattggacctaaaaaacgtataaacccttaggccacggctagggcacctaaggaaagttgttatgcgtccctggattcaacattatcattttaaaaatatttccagtgccgattatttgcatgtgtttgaacccaataacgtgtatgccgctgccggtaatcgacgcagggacctaagttgtaatagtaaaatttttataaaggtcacggctaataaacgtacacaaacttgggttattgcacatacggcgacggccattagcgaccggcatggtgcccttgtttgaaggtattgtgaactatgatgaaattattcaagaagtgcgggacaccgatcattttgccagtttggaatggtttggcctggccgtaccacgggaacaaacttccataacacttgatactactttaataagttcttcacgccctgtggctagtaaaacggtcaaaccttaccaaaccgcataacgccaaagatattttgaaagccgaaatgaaagtgctgaccaatcggaatttggaagtggtgacctcctaatttttggggatcaattcgagctcgggtattgcggtttctataaaactttcggctttactttcacgactggttagccttaaaccttcaccactggaggattaaaaacccctagttaagctcgagccttcccaaactagtatgtagggtgaggttatagctatgcagaatcccattaatgatttgcggtccgccattgccttgttgcaacggcatcccggtcattataagggtttgatcatacatcccactccaatatcgatacgtcttagggtaattactaaacgccaggcggtaacggaacaacgttgccgtagggccagtaataattgaaaccgatcatcccgttgatcccaatgccgaattagccggtgtgtatcggcatattggtgccggtggcaccgtgaaacgtcccacccgtaccggtctaactttggctagtagggcaactagggttacggcttaatcggccacacatagccgtataaccacggccaccgtggcactttgcagggtgggcatggccagccgctatgatgtttaattccgtgaaaggctatcccggttcccggattttggtgggtatgcatgccagtcgggaacgggctgccttgttgttgggctgtgtggcgatactacaaattaaggcactttccgatagggccaagggcctaaaaccacccatacgtacggtcagcccttgcccgacggaacaacaacccgacacagccctccaaattggcccaacatgtgggtcaagccgtgaaaaatcccgtggctcccgtggtggttcccgcttcccaagctccctgtcaagaacaagtgtttcgggaggtttaaccgggttgtacacccagttcggcactttttagggcaccgagggcaccaccaagggcgaagggttcgagggacagttcttgttcacaaatatgccgatgatcccgattttgatctgcggaaattgttacccgctcccaccaatacccccattgatgccggtccctttttttgtttgggcttggtgttggatacggctactagggctaaaactagacgcctttaacaatgggcgagggtggttatgggggtaactacggccagggaaaaaaacaaacccgaaccacaacccctccgatcccgaagatacctccttgaccgatgtgaccattcatcggttgtgtgtgcaagaacgggatgaattgtccatgtttttggctgccggtcggcaggaggctagggcttctatggaggaactggctacactggtaagtagccaacacacacgttcttgccctacttaacaggtacaaaaaccgacggccagccgttattgaagtgtttcggaaaaaagccgaagccgctggcaaacccttgcccgtgaccattaatatgggcttagatcccgctatttacattggtgcctgttttataacttcacaaagccttttttcggcttcggcgaccgtttgggaacgggcactggtaattatacccgaatctagggcgataaatgtaaccacggacaaaagaagctcccaccaccccctttggctataatgaattgggcgttgccggtgccttgcggcaacaacccgtggaattggtgcaaggcgtggccgtgaaagaaacttcgagggtggtgggggaaaccgatattacttaacccgcaacggccacggaacgccgttgttgggcaccttaaccacgttccgcaccggcactttctttaagccattgcccgtgccgaaattattattgaaggcgaattattacccggtgtgcgggtgcgggaagatcaacataccaataccggtcatgccatgcccgattcggtaacgggcacggctttaataataacttccgcttaataatgggccacacgcccacgcccttctagttgtatggttatggccagtacggtacgggctatttcccggttattgtggcgaagccaatcccagtttgcccgtgattaaagtgaaagccgtgaccatgcggaatcatgccattttgcaaaccttggtgggttaaagggccaataacaccgcttcggttagggtcaaacgggcactaatttcactttcggcactggtacgccttagtacggtaaaacgtttggaaccacccacccggtgaagaacataccaccttagccggtttgcccaccgaagcctccattcggaatgccgtggaagaagccattcccggttttttgcaaaatgtgtatggggccacttcttgtatggtggaatcggccaaacgggtggcttcggaggtaagccttacggcaccttcttcggtaagggccaaaaaacgttttacacataccccataccgctggcggtggcaaatttttgggcattttgcaagtgaaaaaacggcaaccctccgatgaaggtcggcaaggccaagccgctttgattgccttgggtatggcgaccgccaccgtttaaaaacccgtaaaacgttcacttttttgccgttgggaggctacttccagccgttccggttcggcgaaactaacggaaggccacctattccgaattgaaaaatattattttggtggatgaagatgtggatatttttgactccgatgatattttgtgggccatgaccacccggatgcaaccggtggataaggcttaactttttataataaaaccacctacttctacacctataaaaactgaggctactataaaacacccggtactggtgggcctacgttggtgatgtgtccattaccaccttacccggaattcggggtcatcaattagatccctcccaaagtcccgattattccacctccattcggggtaatggcatttccactacacaggtaatggtggaatgggccttaagccccagtagttaatctagggagggtttcagggctaataaggtggaggtaagccccattaccgtaaacctgtaaaaccatttttgattgtaccgtgccctgggccttgaaagcccgttttgaacgggctccctttatggaagttgatcccaccccctgggctcccgaggacattttggtaaaaactaacatggcacgggacccggaactttcgggcaaaacttgcccgagggaaataccttcaactagggtgggggacccgagggctattgttttccgataaaaaataatttttggggatcaattcgagctcggtacccaaactagtatgtagggtgaggttatagctatgaccgctgccaatgtgataacaaaaggctattttttattaaaaacccctagttaagctcgagccatgggtttgatcatacatcccactccaatatcgatactggcgacggttacactaaattttgaataccgaagaagtgcaaaattttattaacttgttgtccggcttggaacaagaaggcggtaatccccgtattaaacaaattattcatcgggttttaaaacttatggcttcttcacgttttaaaataattgaacaacaggccgaaccttgttcttccgccattaggggcataatttgtttaataagtagcccaggtgtccgacctgtttaaatccattgaagatctggaaattacctccgatgaatattgggctgccattgcctatttgaatcaattgggcacctcccatgaaccacaggctggacaaatttaggtaacttctagacctttaatggaggctacttataacccgacggtaacggataaacttagttaacccgtggagggtacttgccggtttgttgagtcccggtttgggctttgatcattttttggatatgcggatggatgccattgatgctgccttgggcattgataatcccactccccgtacggccaaacaactcagggccaaacccgaaactagtaaaaaacctatacgcctacctacggtaactacgacggaacccgtaactattagggtgaggggcatccattgaaggtcccttgtatgttgccggtgctcccgtgtcccaaggctttgcccgtatggatgatggctccgatcccaatggccataccttgattttgcaggtaacttccagggaacatacaacggccacgagggcacagggttccgaaacgggcatacctactaccgaggctagggttaccggtatggaactaaaacgttggcaccatttataatgccgatggtcaacccattcccaatgcccaagtggaaatttggcacgccaataccaaaggcttttactcccattttgatcccaccaccgtggtaaatattacggctaccagttgggtaagggttacgggttcacctttaaaccgtgcggttatggtttccgaaaatgagggtaaaactagggtggggtgaacaaaccccctttaatatgcgtcggaccattatgaccgatgcccaaggccattatcgggtgcaaaccattttgccctccggctatggctgtcctcccacttgtttgggggaaattatacgcagcctggtaatactggctacgggttccggtaatagcccacgtttggtaaaacgggaggccgataccgacaggagccaatggtcccacccaacaattgttaaatcaattgggtcggcatggcaatcgtcccgctcatattcatttttttgtgagtgccgatggctatcggaaattggttaccagggtgggttgttaacaatttagttaacccagccgtaccgttagcagggcgagtataagtaaaaaaacactcacggctaccgatagcctttaagaccacccaaattaatgtggctggcgatccctatacctatgatgattttgcctttgccacccgtgaaggcttggtggtggaagccattgaacataccgatctggtgggtttaattacaccgaccgctagggatatggatactactaaaacggaaacggtgggcacttccgaaccaccaccttcggtaacttgtatggctacccgctacctcccaacggaatggtgtggaaggtccctttgccgaaatggtgtttgatttgaaattgtcccggttggtggatggcgtggataatcaagtgggggcgatggagggttgccttaccacaccttccagggaaacggctttaccacaaactaaactttaacagggccaaccacctaccgcacctattagttcacctggatcgtccccgtttgcaagcctaatttttggggatcaattcgagctcggtacccaaactagtatgtagggtgaggttatagctatgaatcggcaacaaacctagcaggggcaaacgttcggattaaaaacccctagttaagctcgagccatgggtttgatcatacatcccactccaatatcgatacttagccgttgttattgatgccttggtgaaacaaatgaatgtggataccgccaaaggtcccgtggatgaacggattcaacaagtggtggtgcggttgttgggcgatttgtttctaactacggaaccactttgtttacttacacctatggcggtttccagggcacctacttgcctaagttgttcaccaccacgccaacaacccgctaaacaaagaggccattgaagatttagatattcaaccctccgaagtgtggaaaggcttggaatatttgaccgatgccggtcaagccaatgaattgggtttgttagccgctccggtaacttctaaatctataagttgggaggcttcacacctttccgaaccttataaactggctacggccagttcggttacttaacccaaacaatcggcgtggcttgggcttggaacattatttagatttgcgtgccgatgaagccgatgccaaagccggtattaccggtggtactccccgtactattgaaggacccctgaccgaacccgaaccttgtaataaatctaaacgcacggctacttcggctacggtttcggccataatggccaccatgaggggcatgataacttcctggggactacgtggctggcgctcccgaatccgtgggctttgctcggatggacgatggtagtgaatccgataaagtggataccttgattattgaagggaccgtgaccgatgcaccgaccgcgagggcttaggcacccgaaacgagcctacctgctaccatcacttaggctatttcacctatggaactaataacttccctggcactggcataccgaaggcaacattattgaaggtgccaaagtggaagtgtggcatgccaacagtttgggcaattattccttttttgataaatcccaatccgattttaatatggcttccgttgtaataacttccacggtttcaccttcacaccgtacggttgtcaaacccgttaataaggaaaaaactatttagggttaggctaaaattcctgcgtcgcaccattttaaccgacgtgaatggcaaatatgtggccttgaccaccatgcccgtgggctacggttgtcctcccgaaggcaccacccaagccggacgcagcgtggtaaaattggctgcacttaccgtttatacaccggaactggtggtacgggcacccgatgccaacaggagggcttccgtggtgggttcggttgttgaataaattaggccgtcacggtaatcgtccctcccatgtgcattattttgtgtccgctcccggttatcgcaaattaaccactcagtttaacattgaacaacttatttaatccggcagtgccattagcagggagggtacacgtaataaaacacaggcgagggccaatagcgtttaattggtgagtcaaattgtaacaaggcgacgaatatctgtgggatgactttgcttttgctactcgggatggtttggtggccaccgccaccgatgttaccgacgaagccgaaattgcccgtcgttccgctgcttatagacaccctactgaaacgaaaacgatgagccctaccaaaccaccggtggcggtggctacaatggctgcttcggctttaacgggcagcggaattggataaaccctttaaacatattacctttaatgtggaactggtgaaagaagccgaagctgctccctcctccgaagtggaacggcgtcgtgcctccccttaacctatttgggaaatttgtataatggaaattacaccttgaccactttcttcggcttcgacgagggaggaggcttcaccttgccgcagcacggagggcctaaggtaccggatcctaattccttggtgtaatgccaactgaataatctgcaaattgcactctccttcaatggggggtgctttttgcttgactgagtacggattccatggcctaggattaaggaaccacattacggttgacttattagacgtttaacgtgagaggaagttaccccccacgaaaaacgaactgactcatatcttctgattgctgatcttgattgccatcgatcgccggggagtccggggcagttaccattagagagtctagagaattaatccatcttcgatagaggaattagaagactaacgactagaactaacggtagctagcggcccctcaggccccgtcaatggtaatctctcagatctcttaattaggtagaagctatctccttatatgggggaagaacctgtgccggcggataaagcattaggcaagaaattcaagaaaaaaaatgcctcctggagcattgaagaaagcgaagctctgtaccggatacccccttcttggacacggccgcctatttcgtaatccgttctttaagttcttttttttacggaggacctcgtaacttctttcgcttcgagacatggccgttgaggcctggggggcaccttattttgccattaatgccgctggtaacataaccgtctctcccaacggcgatcggggcggttcgttagatttgttggaaccaactccggaccccccgtggaataaaacggtaattacggcgaccattgtattggcagagagggttgccgctagccccgccaagcaatctaaacaaccttgtggtggaagccctgcggcaaagaaagctcggcttacccctattaattcgtttttccgatattttggccgatcgcctagagcgattgaatagttgttttgcaccaccttcgggacgccgtttctttcgagccgaatggggataattaagcaaaaaggctataaaaccggctagcggatctcgctaacttatcaacaaaacgcaaggcgatcgaattcgtaatcatggtcatagctgtttcctgtgtgaaattgttatccgctcacaattccacacaacatacgagccggaagcataaagtggttccgctagcttaagcattagtaccagtatcgacaaaggacacactttaacaataggcgagtgttaaggtgtgttgtatgctcggccttcgtatttcactaaagcctggggtgcctaatgagtgagctaactcacattaattgcgttgcgctcactgcccgctttccagtcgggaaacctgtcgtgccagctgcattaaatttcggaccccacggattactcactcgattgagtgtaattaacgcaacgcgagtgacgggcgaaaggtcagccctttggacagcacggtcgacgtaatttgaatcggccaacgcgcggggagaggcggtttgcgtattgggcgcacttagccggttgcgcgcccctctccgccaaacgcataacccgcg